SEQUENCE LISTING (35 sequences)

SEQ ID NO: 1 - protein, 5 aa, Artificial
  CDRH1
DYAMH

SEQ ID NO: 2 - protein, 17 aa, Artificial
  CDRH2
LISIYSDHTNYNQKFQG

SEQ ID NO: 3 - protein, 12 aa, Artificial
  CDRH3
NNYGNYGWYFDV

SEQ ID NO: 4 - protein, 10 aa, Artificial
  CDRL1
SASSSVSYMH

SEQ ID NO: 5 - protein, 7 aa, Artificial
  CDRL2
DTSKLAS

SEQ ID NO: 6 - protein, 9 aa, Artificial
  CDRL3
FQGSGYPYT

SEQ ID NO: 7 - protein, 121 aa, Artificial
  Humanized Heavy Chain (VH) Variable Region (H2)
QVQLVQSGAEVKKPGSSVKVSCKASGYTFTDYAMHWVRQAPGQGLEWMGLISIYSDHTNYNQKFQGRVTITADKSTSTAYMELSSLRSEDTAVYYCGRNNYGNYGWYFDVWGQGTTVTVSS

SEQ ID NO: 8 - protein, 106 aa, Artificial
  Humanized Light Chain (VL) Variable Region (L5)
EIVLTQSPATLSLSPGERATLSCSASSSVSYMHWYQQKPGQAPRLLIYDTSKLASGIPARFSGSGSGTDYTLTISSLEPEDFAVYYCFQGSGYPYTFGQGTKLEIK

SEQ ID NO: 9 - protein, 168 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKM

SEQ ID NO: 10 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 11 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 12 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 13 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 14 - protein, 10 aa, Artificial
  37A10S713 VH CDR1
GFTFSDYWMD

SEQ ID NO: 15 - protein, 17 aa, Artificial
  37A10S713 VH CDR2
NIDEDGSITEYSPFVKG

SEQ ID NO: 16 - protein, 8 aa, Artificial
  37A10S713 VH CDR3
WGRFGFDS

SEQ ID NO: 17 - protein, 15 aa, Artificial
  37A10S713 VL CDR1
KSSQSLLSGSFNYLT

SEQ ID NO: 18 - protein, 7 aa, Artificial
  37A10S713 VL CDR2
YASTRHT

SEQ ID NO: 19 - protein, 9 aa, Artificial
  37A10S713 VL CDR3
HHHYNAPPT

SEQ ID NO: 20 - protein, 116 aa, Artificial
  37A10S713 heavy chain variable region
EVQLVESGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 21 - protein, 111 aa, Artificial
  37A10S713 light chain variable region
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 22 - protein, 121 aa, Artificial
  ICOS.33 IgG1f S267E Heavy Chain Variable Domain
EVQLVESGGGLVKPGGSLRLSCAASGFTFSDYFMHWVRQAPGKGLEWVGVIDTKSFNYATYYSDLVKGRFTISRDDSKNTLYLQMNSLKTEDTAVYYCTATIAVPYYFDYWGQGTLVTVSS

SEQ ID NO: 23 - protein, 106 aa, Artificial
  ICOS.33 IgG1f S267E Light Chain Variable Domain
DIQMTQSPSSLSASVGDRVTITCQASQDISNYLSWYQQKPGKAPKLLIYYTNLLAEGVPSRFSGSGSGTDFTFTISSLQPEDIATYYCQQYYNYRTFGPGTKVDIK

SEQ ID NO: 24 - protein, 124 aa, Artificial
  STIM003 Heavy chain variable domain
EVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSS

SEQ ID NO: 25 - protein, 108 aa, Artificial
  STIM003 Light chain variable domain
EIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIK

SEQ ID NO: 26 - protein, 125 aa, Artificial
  XENP23104 [ICOS]_H0.66_L0 Heavy chain variable domain
QVQLVQSGAEVKKPGASVKVSCKASGYTFTGYYMHWVRQAPGQGLEWMGWINPHSGETIYAQKFQGRVTMTRDTSISTAYMELSSLRSEDTAVYYCARTYYYDTSGYYHDAFDVWGQGTMVTVSS

SEQ ID NO: 27 - protein, 5 aa, Artificial
  XENP23104 [ICOS]_H0.66_L0 VH CDR1
GYYMH

SEQ ID NO: 28 - protein, 17 aa, Artificial
  XENP23104 [ICOS]_H0.66_L0 VH CDR2
WINPHSGETIYAQKFQG

SEQ ID NO: 29 - protein, 16 aa, Artificial
  XENP23104 [ICOS]_H0.66_L0 VH CDR3
TYYYDTSGYYHDAFDV

SEQ ID NO: 30 - protein, 107 aa, Artificial
  XENP23104 [ICOS]_H0.66_L0 Light chain variable domain
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIK

SEQ ID NO: 31 - protein, 11 aa, Artificial
  XENP23104 [ICOS]_H0.66_L0 VL CDR1
RASQGISRLLA

SEQ ID NO: 32 - protein, 7 aa, Artificial
  XENP23104 [ICOS]_H0.66_L0 VL CDR2
VASSLQS

SEQ ID NO: 33 - protein, 9 aa, Artificial
  XENP23104 [ICOS]_H0.66_L0 VL CDR3
QQANSFPWT

SEQ ID NO: 34 - protein, 448 aa, Artificial
  Humanized heavy chain (H2L5 IgG4PE)
QVQLVQSGAEVKKPGSSVKVSCKASGYTFTDYAMHWVRQAPGQGLEWMGLISIYSDHTNYNQKFQGRVTITADKSTSTAYMELSSLRSEDTAVYYCGRNNYGNYGWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 213 aa, Artificial
  Humanized light chain (H2L5 IgG4PE)
EIVLTQSPATLSLSPGERATLSCSASSSVSYMHWYQQKPGQAPRLLIYDTSKLASGIPARFSGSGSGTDYTLTISSLEPEDFAVYYCFQGSGYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2021/0395367 A1
(43) Pub. Date: Dec. 23, 2021

(54) DOSING

(71) Applicant: Glaxosmithkline Intellectual Property Development Limited, Middlesex (GB)

(72) Inventors: Elaine Marie PAUL, Collegeville, PA (US); Patrick MAYES, Collegeville, PA (US); Catherine E. ELLIS, Collegeville, PA (US); Jessica KATZ, Collegville, PA (US)

(21) Appl. No.: 17/287,144

(22) PCT Filed: Oct. 21, 2019
(86) PCT No.: PCT/US2019/057257

§ 371 Date: Apr. 21, 2021

Related U.S. Application Data
(60) Provisional application No. 62/902,444, filed on Sep. 19, 2019.
(60) Provisional application No. 62/895,299, filed on Sep. 3, 2019.
(60) Provisional application No. 62/837,385, filed on Apr. 23, 2019.
(60) Provisional application No. 62/807,897, filed on Feb. 20, 2019.
(60) Provisional application No. 62/748,595, filed on Oct. 22, 2018.

(51) Int. Cl. C07K 16/28; A61P 35/00

(52) U.S. Cl. CPC C07K 16/2818; A61K 2039/505; A61P 35/00

(57) ABSTRACT

The present invention relates to a method of treating cancer comprising administering to the human an ICOS binding protein or antigen binding portion thereof at a dose of about 0.08 mg to about 240 mg.

### CROSS-REFERENCE TO RELATED APPLICATIONS

This application claims benefit of U.S. Provisional Application Nos. 62/748,595, filed Oct. 22, 2018, 62/807,897, filed Feb. 20, 2019, 62/837,385, filed Apr. 23, 2019, 62/895,229, filed Sep. 3, 2019, and 62/902,444, filed Sep. 19, 2019, the disclosures of which are incorporated herein by reference in their entirety.

### FIELD OF THE INVENTION

The present invention relates to a method of treating cancer in a mammal. In particular, the present invention relates to dosing of anti-ICOS antibodies and dosing of combinations of anti-ICOS antibodies and PD1 antagonists.

### BACKGROUND TO THE INVENTION

Effective treatment of hyperproliferative disorders, including cancer, is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes includes abnormalities in signal transduction pathways and response to factors that differ from those found in normal cells.

Immunotherapies are one approach to treat hyperproliferative disorders. A major hurdle that scientists and clinicians have encountered in the development of various types of cancer immunotherapies has been to break tolerance to self antigen (cancer) in order to mount a robust anti-tumor response leading to tumor regression. Unlike traditional development of small and large molecule agents that target the tumor, cancer immunotherapies may, among other things, target cells of the immune system that have the potential to generate a memory pool of effector cells to induce more durable effects and minimize recurrences.

Though there have been many recent advances in the treatment of cancer, there remains a need for more effective and/or enhanced treatment of an individual suffering the effects of cancer. The methods herein that relate to combining therapeutic approaches for enhancing anti-tumor immunity address this need.

### SUMMARY OF THE INVENTION

In one aspect, there is provided a method of treating cancer comprising administering to a human an ICOS binding protein or antigen binding portion thereof at a dose of about 0.08 mg to about 240 mg.

In one aspect, a method of treating cancer in a human in need thereof is provided, the method comprising administering to the human an agonist ICOS binding protein or antigen binding portion thereof at a dose of about 0.08 mg to about 240 mg.

In another aspect, an agonist ICOS binding protein or antigen binding portion thereof for use in treating cancer is provided, wherein the ICOS binding protein or antigen binding portion thereof is to be administered at a dose of about 0.08 mg to about 240 mg.

In one aspect, use of an agonist ICOS binding protein or antigen binding portion thereof in the manufacture of a medicament for treating cancer is provided, wherein the agonist ICOS binding protein or antigen binding portion thereof is to be administered at a dose of about 0.08 mg to about 240 mg.

In one aspect, there is provided a pharmaceutical kit comprising about 0.08 mg to about 240 mg of an ICOS binding protein or antigen binding portion thereof.

### DESCRIPTION OF DRAWINGS/FIGURES

FIG. 1 is a diagram showing the Study design.

FIG. 2 is a table showing patient disposition by cohort and dose.

FIG. 3 is a table showing patient and disease characteristics.

FIG. 4 is a table showing treatment-related adverse events (AEs) (in ≥3 patients).

FIGS. 5A-5C are plots showing duration of Study treatment: individual patient data. FIG. 5A shows monotherapy dose escalation cohort. FIG. 5B shows PK/PD cohort. FIG. 5C shows combination dose escalation cohort.

FIGS. 6A-6B are plots showing PK and receptor occupancy. FIG. 6A shows dose-proportional PK from 0.01 mg/kg to 3 mg/kg; no PK difference between monotherapy and combination with pembrolizumab. FIG. 6B shows peak receptor occupancy corresponding to H2L5 IgG4PE maximum plasma concentration; similar relationship for CD8+ receptor occupancy (data not shown).

FIG. 7 is a set of scans of Patient 1 (H2L5 IgG4PE monotherapy treatment).

FIG. 8 is set of scans of Patient 2 (H2L5 IgG4PE plus pembrolizumab combination therapy).

FIG. 9 is a set of scans of a squamous NSCLC patient (H2L5 IgG4PE/pembrolizumab combination therapy).

FIGS. 10A-10C are plots showing results of the pharmacokinetics study. FIG. 10A shows dose-proportional pharmacokinetic (PK) from 0.01 mg/kg to 3 mg/kg; no PK difference between monotherapy and combination with pembrolizumab; Part 1A: N=2 (0.01), 8 (0.03), 15 (0.1), 28 (0.3), 88 (1.0), 16 (3.0). Part 2A: N=5 (0.01), 5 (0.03), 5 (0.1), 163 (0.3), 21 (1.0), and 8 (3.0). FIG. 10B shows peak CD4⁺ Receptor Occupancy (RO) corresponding to H2L5 IgG4PE maximum plasma concentration; similar relationship for CD8⁺ receptor occupancy (data not shown); N=5 (0.03), 14 (0.1), 24 (0.3), 77 (1.0), 11 (3.0), and 5 (10). FIG. 10C shows CD4⁺RO with H2L5 IgG4PE 0.3 mg/kg and 1.0 mg/kg monotherapy versus combination with pembrolizumab over two dosing intervals; Part 1A (monotherapy), Part 2A (pembrolizumab combination).

FIG. 11 is a plot showing receptor occupancy (RO) H2L5 IgG4PE concentration FIGS. 12A-12C are plots showing exposure-response characterization. Regression of: FIG. 12A—best overall response; FIG. 12B—disease control rate; and FIG. 12C—observed percentage change from baseline in tumor sum of longest diameters (SLD); at 9 weeks by H2L5 IgG4PE exposure in HNSCC dose escalation and expansion cohorts illustrates a weak association that is not statistically significant (all regression p-values >0.05). AUC, area under curve; HNSCC, head and neck squamous cell carcinoma; ORR, overall response rate; DCR, disease control rate; SLD, sum of longest diameters.

FIG. 13 is plot showing cytotoxic T cell to Treg Ratio. A potentially favourable CD8:Treg ratio at week 6 on-treatment compared to pre-treatment samples was observed at ICOS exposure of 1000-10000 ng/mL and H2L5 IgG4PE ˜0.3-1.0 mg/kg; ICOS (inducible T cell co-stimulator), Treg (regulatory T cell); Cytotoxic T cell defined as CD3⁺CD8⁺; regulatory T cell defined as CD3⁺CD4⁺FOXP3⁺.

FIGS. 14A-14B are plots showing dose-response analyses. FIG. 14A shows MultiOmyx dose-response curves (N=43 subjects, 40 phenotypes). FIG. 14B shows ratio of cytotoxic T cell proliferation:Treg proliferation. CR, complete response; DC; disease control; DCR, disease control rate (CR+PR+SD ≥18 weeks); ICOS, inducible T cell co-stimulator; ITT, intent-to treat; N, no; PR, partial response; SD, stable disease); Y, yes.

FIG. 15 is a schematic of the study design. *For ≤2 years or until disease progression or unacceptable toxicity; ^{†}subjects enrolled in the Part 2B cohorts (H2L5 IgG4PE/pembrolizumab combination) may be stratified by PD-L1 IHC status and prior PD-1/L1 treatment; ^{†}a subset of HNSCC subjects will be randomly assigned to one of 3 doses of H2L5 IgG4PE in combination with 200 mg of pembrolizumab. IHC, immunohistochemistry; IV, intravenous; Q3W, every 3 weeks

FIGS. 16A-16B are plots showing best tumor response. FIG. 16A shows monotherapy cohort; Patients from both DE and CE phases included. FIG. 16B shows combination cohort; Patients (non-randomised) from both DE and CE phases included. irCR, immune-related complete response; irPD, immune-related progressive disease; irPR, immune-related partial response; irSD, immune-related stable disease; NE, not evaluable; pem, pembrolizumab.

FIGS. 17A-17B are plots showing change from baseline in tumour measurement for FIG. 17A Monotherapy cohort (*PD-1/L1 experienced subjects; →treatment ongoing; ^{†}patients from both DE and CE phases included); and FIG. 17B combination cohort (^{†}Patients from both DE and CE phases included; →treatment ongoing).

FIG. 18 is a chart showing progression-free survival (PFS) for combination therapy.

FIG. 19 is a chart showing overall survival (OS) for combination therapy.

FIGS. 20A-20B are bar graphs showing treatment-related adverse events reported in ≥5% of patients. FIG. 20A shows monotherapy cohort (Part 1A and 1B). FIG. 20B shows combination cohort (Part 2A and 2B).

FIG. 21 shows PD-L1 immunohistochemistry (BOR, best overall response; CPS, combined positive score; CR, complete response; NE, non-evaluable; PD, progressive disease; PR, partial response; SD, stable disease). The filled circle denotes CPS<1 was imputed to 0.5 to enable graphing. Area between the dashed lines indicates 1≤CPS <20.

FIG. 22 shows a set of scans of an HNSCC patient case study for combination therapy (H2L5 IgG4PE plus pembrolizumab combination therapy).

FIG. 23 is a set of scans of an HNSCC patient case study for H2L5 IgG4PE monotherapy.

### DETAILED DESCRIPTION OF THE INVENTION

Antigen Binding Proteins and Antibodies that Bind ICOS

“Antigen Binding Protein (ABP)” means a protein that binds an antigen, including antibodies or engineered molecules that function in similar ways to antibodies. Such alternative antibody formats include triabody, tetrabody, miniantibody, and a minibody. Also included are alternative scaffolds in which the one or more CDRs of any molecules in accordance with the disclosure can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain. An ABP also includes antigen binding fragments of such antibodies or other molecules. Further, an ABP may comprise the VH regions of the invention formatted into a full length antibody, a (Fab′)2 fragment, a Fab fragment, a bi-specific or biparatopic molecule or equivalent thereof (such as scFV, bi- tri- or tetra-bodies, Tandabs, etc.), when paired with an appropriate light chain. The ABP may comprise an antibody that is an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. The ABP may also be a chimeric antibody of the type described in WO86/01533, which comprises an antigen binding region and a non-immunoglobulin region. The terms “ABP,” “antigen binding protein,” and “binding protein” are used interchangeably herein.

As used herein “ICOS” means any Inducible T-cell costimulator protein. Pseudonyms for ICOS (Inducible T-cell COStimulator) include AILIM; CD278; CVID1, JTT-1 or JTT-2, MGC39850, or 8F4. ICOS is a CD28-superfamily costimulatory molecule that is expressed on activated T cells. The protein encoded by this gene belongs to the CD28 and CTLA-4 cell-surface receptor family. It forms homodimers and plays an important role in cell-cell signaling, immune responses, and regulation of cell proliferation. The amino acid sequence of human ICOS (isoform 2) (Accession No.: UniProtKB—Q9Y6W8-2) is shown below as SEQ ID NO:9.

(SEQ ID NO: 9)
MKSGLWYFFLFCLRIKVLTGEINGSANYEMFIFHNGGVQILCKYPDIVQ
QFKMQLLKGGQILCDLTKTKGSGNTVSIKSLKFCHSQLSNNSVSFFLYN
LDHSHANYYFCNLSIFDPPPFKVTLTGGYLHIYESQLCCQLKFWLPIGC
AAFWVCILGCILICWLTKKM

The amino acid sequence of human ICOS (isoform 1) (Accession No.: UniProtKB—Q9Y6W8-1) is shown below as SEQ ID NO: 10.

(SEQ ID NO: 10)
MKSGLWYFFLFCLRIKVLTGEINGSANYEMFIFHNGGVQILCKYPDIVQ
QFKMQLLKGGQILCDLTKTKGSGNIVSIKSLKFCHSQLSNNSVSFFLYN
LDHSHANYYFCNLSIFDPPPFKVTLTGGYLHIYESQLCCQLKFWLPIGC
AAFWVCILGCILICWLTKKKYSSSVHDPNGEYMFMRAVNTAKKSRLTDV
TL

Activation of ICOS occurs through binding by ICOS-L (B7RP-1/B7-H2). Neither B7-1 nor B7-2 (ligands for CD28 and CTLA4) bind or activate ICOS. However, ICOS-L has been shown to bind weakly to both CD28 and CTLA-4 (Yao S et al., “B7-H2 is a costimulatory ligand for CD28 in human”, Immunity, 34(5); 729-40 (2011)). Expression of ICOS appears to be restricted to T cells. ICOS expression levels vary between different T cell subsets and on T cell activation status. ICOS expression has been shown on resting TH17, T follicular helper (TFH) and regulatory T (Treg) cells; however, unlike CD28; it is not highly expressed on naïve TH1 and TH2 effector T cell populations (Paulos C M et al., “The inducible costimulator (ICOS) is critical for the development of human Th17 cells”, Sci Transl Med, 2(55); 55ra78 (2010)). ICOS expression is highly induced on CD4+ and CD8+ effector T cells following activation through TCR engagement (Wakamatsu E, et al., “Convergent and divergent effects of costimulatory molecules in conventional and regulatory CD4+ T cells”, Proc Natl Acad Sci USA, 110(3); 1023-8 (2013)). Co-stimulatory signalling through ICOS receptor only occurs in T cells receiving a concurrent TCR activation signal (Sharpe A H and Freeman G J. “The B7-CD28 Superfamily”, Nat. Rev Immunol, 2(2); 116-26 (2002)). In activated antigen specific T cells, ICOS regulates the production of both TH1 and TH2 cytokines including IFN-γ, TNF-α, IL-10, IL-4, IL-13 and others. ICOS also stimulates effector T cell proliferation, albeit to a lesser extent than CD28 (Sharpe A H and Freeman G J. “The B7-CD28 Superfamily”, Nat. Rev Immunol, 2(2); 116-26 (2002)). Antibodies to ICOS and methods of using in the treatment of disease are described, for instance, in WO 2012/131004, US20110243929, and US20160215059. US20160215059 is incorporated by reference herein. CDRs for murine antibodies to human ICOS having agonist activity are shown in PCT/EP2012/055735 (WO 2012/131004). Antibodies to ICOS are also disclosed in WO 2008/137915, WO 2010/056804, EP 1374902, EP1374901, and EP1125585. Agonist antibodies to ICOS or ICOS binding proteins are disclosed in WO2012/13004, WO2014/033327, WO2016/120789, US20160215059, and US20160304610. Exemplary antibodies in US2016/0304610 include 37A105713. Sequences of 37A105713 are reproduced below as SEQ ID NOS: 14-21.

37A10S713 V_{H} CDR1:
(SEQ ID NO: 14)
GFTFSDYWMD
37A10S713 V_{H} CDR2:
(SEQ ID NO: 15)
NIDEDGSITEYSPFVKG
37A10S713 V_{H} CDR3:
(SEQ ID NO: 16)
WGRFGFDS
37A10S713 V_{L} CDR1:
(SEQ ID NO: 17)
KSSQSLLSGSFNYLT
37A10S713 V_{L} CDR2:
(SEQ ID NO: 18)
YASTRHT
37A10S713 V_{L} CDR3:
(SEQ ID NO: 19)
HHHYNAPPT
37A10S713 heavy chain variable region:
(SEQ ID NO: 20)
EVQLVESGG LVQPGGSLRL SCAASGFTFS DYWMDWVRQA
PGKGLVWVSN IDEDGSITEY SPFVKGRFTI SRDNAKNTLY
LQMNSLRAED TAVYYCTRWG RFGFDSWGQG TLVTVSS
37A10S713 light chain variable region:
(SEQ ID NO: 21)
DIVMTQSPDS LAVSLGERAT INCKSSQSLL SGSFNYLTWY
QQKPGQPPKL LIFYASTRHT GVPDRFSGSG SGTDFTLTIS
SLQAEDVAVY YCHHHYNAPP TFGPGTKVDI K

Exemplary antibodies in US2018/0289790 include ICOS.33 IgG1f 5267E. Sequences of ICOS.33 IgG1f 5267E are reproduced below as SEQ ID NOS: 22-23:

ICOS.33 IgG1f S267E Heavy Chain Variable Domain
(SEQ ID NO: 22)
EVQLVESGGG LVKPGGSLRL SCAASGFTFS
DYFMHWVRQAPGKGLEWVGV IDTKSFNYAT YYSDLVKGRF
TISRDDSKNT LYLQMNSLKT EDTAVYYCTA TIAVPYYFDY
WGQGTLVTVS S
ICOS.33 IgG1f S267E Light Chain Variable Domain
(SEQ ID NO: 23)
DIQMTQSPSS LSASVGDRVT ITCQASQDIS NYLSWYQQKP
GKAPKLLIYY TNLLAEGVPS RFSGSGSGTD FTFTISSLQP
EDIATYYCQQ YYNYRTFGPG TKVDIK

Exemplary antibodies in WO2018/029474 include STIM003. Sequences of STIM003 are reproduced below as SEQ ID NOS: 24-25.

STIM003 Heavy chain variable domain
(SEQ ID NO: 24)
EVQLVESGGGWRPGGSLRLSCVASGVTFDDYGMSWVRQAPGKGLEWVSG
INWNGGDTDYSDSVKGRFTISRDNAKNSLYLQMNSLRAEDTALYYCARD
FYGSGSYYHVPFDYWGQGILVTVSS
STIM003 Light chain variable domain
(SEQ ID NO: 25)
EIVLTQSPGTLSLSPGERATLSCRASQSVSRSYLAWYQQKRGQAPRLLI
YGASSRATGIPDRFSGDGSGTDFTLSISRLEPEDFAVYYCHQYDMSPFT
FGPGTKVDIK

Exemplary antibodies in WO2018/049497 include XENP23104. Sequences of the ICOS binding Fab side ([ICOS]_H0.66_L0) of XENP23104 are reproduced below as SEQ ID NOS: 26-33.

XENP23104 [ICOS]_H0.66_L0 Heavy chain variable
domain
(SEQ ID NO: 26)
QVQLVQSGAEVKKPGASVKVSCKASGYTFTGYYMHWVRQAPGQGLEWMG
WINPHSGETIYAQKFQGRVTMTRDTSISTAYMELSSLRSEDTAVYYCAR
TYYYDTSGYYHDAFDVWGQGTMVWSS
XENP23104 [ICOS]_H0.66_L0 V_{H} CDR1:
(SEQ ID NO: 27)
GYYMH
XENP23104 [ICOS]_H0.66_L0 V_{H} CDR2:
(SEQ ID NO: 28)
WINPHSGETIYAQKFQG
XENP23104 [ICOS]_H0.66_L0 V_{H} CDR3:
(SEQ ID NO: 29)
TYYYDTSGYYHDAFDV
XENP23104 [ICOS]_H0.66_L0 Light chain variable
domain
(SEQ ID NO: 30)
DIQMTQSPSSVSASVGDRVTITCRASQGISRLLAWYQQKPGKAPKLLIY
VASSLQSGVPSRFSGSGSGTDFTLTISSLQPEDFATYYCQQANSFPWTF
GQGTKVEIK
XENP23104 [ICOS]_H0.66_L0 V_{L} CDR1:
(SEQ ID NO: 31)
RASQGISRLLA
XENP23104 [ICOS]_H0.66_L0 V_{L} CDR2:
(SEQ ID NO: 32)
VASSLQS
XENP23104 [ICOS]_H0.66_L0 V_{L} CDR3:
(SEQ ID NO: 33)
QQANSFPWT

By “agent directed to ICOS” is meant any chemical compound or biological molecule capable of binding to ICOS. In some embodiments, the agent directed to ICOS is an ICOS binding protein. In some other embodiments, the agent directed to ICOS is an ICOS agonist. In some embodiments, the ICOS binding protein is an agonist ICOS binding protein.

The term “ICOS binding protein” as used herein refers to antibodies and other protein constructs, such as domains, which are capable of binding to ICOS. In some instances, the ICOS is human ICOS. The term “ICOS binding protein” can be used interchangeably with “ICOS antigen binding protein.” Thus, as is understood in the art, anti-ICOS antibodies and/or ICOS antigen binding proteins would be considered ICOS binding proteins. As used herein, “antigen binding protein” is any protein, including but not limited to antibodies, domains and other constructs described herein, that binds to an antigen, such as ICOS. As used herein “antigen binding portion” of an ICOS binding protein would include any portion of the ICOS binding protein capable of binding to ICOS, including but not limited to, an antigen binding antibody fragment.

In one embodiment, the ICOS antibodies of the present invention comprise any one or a combination of the following CDRs:

CDRH1:
(SEQ ID NO: 1)
DYAMH
CDRH2:
(SEQ ID NO: 2)
LISIYSDHTNYNQKFQG
CDRH3:
(SEQ ID NO: 3)
NNYGNYGWYFDV
CDRL1:
(SEQ ID NO: 4)
SASSSVSYMH
CDRL2:
(SEQ ID NO: 5)
DTSKLAS
CDRL3:
(SEQ ID NO: 6)
FQGSGYPYT

In some embodiments, the anti-ICOS antibodies of the present invention comprise a heavy chain variable region having at least 90% sequence identity to SEQ ID NO:7. Suitably, the ICOS binding proteins of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:7.

Humanized Heavy Chain (V_{H}) Variable Region (H2):
(SEQ ID NO: 7)
QVQLVQSGAE VKKPGSSVKV SCKASGYTFT DYAMHWVRQA
PGQGLEWMGL ISIYSDHTNY NQKFQGRVTI TADKSTSTAY
MELSSLRSED TAVYYCGRNN YGNYGWYFDV WGQGTIVIVS
S

In one embodiment of the present invention the ICOS antibody comprises CDRL1 (SEQ ID NO:4), CDRL2 (SEQ ID NO:5), and CDRL3 (SEQ ID NO:6) in the light chain variable region having the amino acid sequence set forth in SEQ ID NO:8. ICOS binding proteins of the present invention comprising the humanized light chain variable region set forth in SEQ ID NO:8 are designated as “L5.” Thus, an ICOS binding protein of the present invention comprising the heavy chain variable region of SEQ ID NO:7 and the light chain variable region of SEQ ID NO:8 can be designated as H2L5 herein.

In some embodiments, the ICOS binding proteins of the present invention comprise a light chain variable region having at least 90% sequence identity to the amino acid sequence set forth in SEQ ID NO:8. Suitably, the ICOS binding proteins of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:8.

Humanized Light Chain (V_{L}) Variable Region (L5)
(SEQ ID NO: 8)
EIVLTQSPAT LSLSPGERAT LSCSASSSVS YMHWYQQKPG
QAPRLLIYDT SKLASGIPAR FSGSGSGTDY TLTISSLEPE
DFAVYYCFQG SGYPYTFGQG TKLEIK

In one embodiment, the ICOS binding protein is a humanized monoclonal antibody comprising a heavy chain amino acid sequence having at least 90%, 91%, 92,%, 93%, 94%, 95%, 96%, 97%, 98%, 99% or 100% sequence identity to the amino acid sequence set forth in SEQ ID NO: 34.

(SEQ ID NO: 34)
QVQLVQSGAEVKKPGSSVKVSCKASGYTFTDYAMHWVRQAPGQGLEWMG
LISIYSDHTNYNQKFQGRVTITADKSTSTAYMELSSLRSEDTAVYYCGR
NNYGNYGWYFDVWGQGTTVTVSSASTKGPSVFPLAPCSRSTSESTAALG
CLVKDYFPEPVTVSWNSGALTSGVHTFPAVLQSSGLYSLSSVVTVPSSS
LGTKTYTCNVDHKPSNTKVDKRVESKYGPPCPPCPAPEFEGGPSVFLFP
PKPKDTLMISRTPEVTCVVVDVSQEDPEVQFNWYVDGVEVHNAKTKPRE
EQFNSTYRWSVLTVLHQDWLNGKEYKCKVSNKGLPSSIEKTISKAKGQP
REPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSL
SLSLGK

In one embodiment, the ICOS binding protein is a humanized monoclonal antibody comprising a light chain amino acid sequence having at least 90%, 91%, 92,%, 93%, 94%, 95%, 96%, 97%, 98%, 99% or 100% sequence identity to the amino acid sequence set forth in SEQ ID NO: 35.

(SEQ ID NO: 35)
EIVLTQSPATLSLSPGERATLSCSASSSVSYMHWYQQKPGQAPRLLIYD
TSKLASGIPARFSGSGSGTDYTLTISSLEPEDFAVYYCFQGSGYPYTFG
QGTKLEIKRTVAAPSVFIFPPSDEQLKSGTASVVCLLNNFYPREAKVQW
KVDNALQSGNSQESVTEQDSKDSTYSLSSTLTLSIKADYEKHKVYACEV
THQGLSSPVTKSFNRGEC

CDRs or minimum binding units may be modified by at least one amino acid substitution, deletion or addition, wherein the variant antigen binding protein substantially retains the biological characteristics of the unmodified protein, such as an antibody comprising SEQ ID NO:7 and SEQ ID NO:8.

It will be appreciated that each of CDR H1, H2, H3, L1, L2, L3 may be modified alone or in combination with any other CDR, in any permutation or combination. In one embodiment, a CDR is modified by the substitution, deletion or addition of up to 3 amino acids, for example 1 or 2 amino acids, for example 1 amino acid. Typically, the modification is a substitution, particularly a conservative substitution (referred herein also as a direct equivalent), for example as shown in Table 1 below.

TABLE 1

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

The subclass of an antibody in part determines secondary effector functions, such as complement activation or Fc receptor (FcR) binding and antibody dependent cell cytotoxicity (ADCC) (Huber, et al., Nature 229(5284): 419-20 (1971); Brunhouse, et al., Mol Immunol 16(11): 907-17 (1979)). In identifying the optimal type of antibody for a particular application, the effector functions of the antibodies can be taken into account. For example, hIgG1 antibodies have a relatively long half life, are very effective at fixing complement, and they bind to both FcγRI and FcγRII. In contrast, human IgG4 antibodies have a shorter half life, do not fix complement and have a lower affinity for the FcRs. Replacement of serine 228 with a proline (S228P) in the Fc region of IgG4 reduces heterogeneity observed with hIgG4 and extends the serum half life (Kabat, et al., “Sequences of proteins of immunological interest” 5.sup.th Edition (1991); Angal, et al., Mol Immunol 30(1): 105-8 (1993)). A second mutation that replaces leucine 235 with a glutamic acid (L235E) eliminates the residual FcR binding and complement binding activities (Alegre, et al., J Immunol 148(11): 3461-8 (1992)). The resulting antibody with both mutations is referred to as IgG4PE. The numbering of the hIgG4 amino acids was derived from EU numbering reference: Edelman, G. M. et al., Proc. Natl. Acad. USA, 63, 78-85 (1969). PMID: 5257969. In one embodiment of the present invention the ICOS antibody is an IgG4 isotype. In one embodiment, the ICOS antibody comprises an IgG4 Fc region comprising the replacement S228P and L235E may have the designation IgG4PE.

As used herein “ICOS-L” and “ICOS Ligand” are used interchangeably and refer to the membrane bound natural ligand of human ICOS. ICOS ligand is a protein that in humans is encoded by the ICOSLG gene. ICOSLG has also been designated as CD275 (cluster of differentiation 275). Pseudonyms for ICOS-L include B7RP-1 and B7-H2.

### PD1 Antagonists

As used herein, an “agent directed to PD-1” or “agent directed to PD1” means any chemical compound or biological molecule capable of binding to PD1. In some embodiments, the agent directed to PD1 is a PD1 antagonist.

The term “PD1 binding protein” or “PD-1 binding protein” as used herein refers to antibodies and other protein constructs, such as domains, which are capable of binding to PD1. In some instances, the PD1 is human PD1. The term “PD1 binding protein” can be used interchangeably with “PD1 antigen binding protein.” Thus, as is understood in the art, anti-PD1 antibodies and/or PD1 antigen binding proteins would be considered PD1 binding proteins. As used herein, “antigen binding protein” is any protein, including but not limited to antibodies, domains and other constructs described herein, that binds to an antigen, such as PD1. As used herein “antigen binding portion” of a PD1 binding protein would include any portion of the PD1 binding protein capable of binding to PD1, including but not limited to, an antigen binding antibody fragment.

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells (Agata et al., supra; Okazaki et al. (2002) Curr. Opin. Immunol 14:391779-82; Bennett et al. (2003) J Immunol 170:711-8) The initial members of the family, CD28 and ICOS, were discovered by functional effects on augmenting T cell proliferation following the addition of monoclonal antibodies (Hutloff et al. (1999) Nature 397:263-266; Hansen et al. (1980) Immunogenics 10:247-260). PD-1 was discovered through screening for differential expression in apototic cells (Ishida et al. (1992) EMBO J 11:3887-95) The other members of the family, CTLA-4, and BTLA were discovered through screening for differential expression in cytotoxic T lymphocytes and TH1 cells, respectively. CD28, ICOS and CTLA-4 all have an unpaired cysteine residue allowing for homodimerization. In contrast, PD-1 is suggested to exist as a monomer, lacking the unpaired cysteine residue characteristic in other CD28 family members. PD-1 antibodies and methods of using in treatment of disease are described in US Patent Nos.: U.S. Pat. Nos. 7,595,048; 8,168,179; 8,728,474; 7,722,868; 8,008,449; 7,488,802; 7,521,051; 8,088,905; 8,168,757; 8,354,509; and US Publication Nos. US20110171220; US20110171215; and US20110271358. Combinations of CTLA-4 and PD-1 antibodies are described in U.S. Pat. No. 9,084,776.

The agent directed to PD1 is a PD1 antagonist and blocks binding of PD-L1 expressed on a cancer cell to PD-1 expressed on an immune cell (T cell, B cell or NKT cell) and may also block binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. Human PD-1 amino acid sequences can be found in NCBI Locus No.: NP_005009. The amino acid sequence in NCBI Locus No.: NP_005009 is reproduced below:

(SEQ ID NO: 11)
MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGWGGLLGSLVLLVWVLAVICSRAAR
GTIGARRTGQPLKEDPSAVPVFSVDYGELDFQWREKTPEPPVPCVPEQT
EYATIVFPSGMGTSSPARRGSADGPRSAQPLRPEDGHCSWPL

Human PD-L1 and PD-L2 amino acid sequences can be found in NCBI Locus No.: NP_054862 and NP_079515, respectively.

The amino acid sequence in NCBI Locus No.:
NP_054862 is reproduced below:
(SEQ ID NO: 12)
MRIFAVFIFMTYWHLLNAFTVTVPKDLYVVEYGSNMTIECKFPVEKQLD
LAALIVYWEMEDKNITQFVHGEEDLKVQHSSYRQRARLLKDQLSLGNAA
LQITDVKLQDAGVYRCMISYGGADYKRITVINNAPYNKINQRILVVDPV
TSEHELTCQAEGYPKAEVIWTSSDHQVLSGKTTTTNSKREEKLFNVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET
The amino acid sequence in NCBI Locus No.:
NP_079515 is reproduced below:
(SEQ ID NO: 13)
MIFELMLSLELQLHQTAALFTVTVPKELYITEHGSNVTLECNFDTGSHV
NLGAITASLQWENDTSPHRERATLLEEQLPLGKASFHIPQVQVRDEGQY
QCIITYGVAWDYKYLTLIWKASYRKINTHILKWETDEVELTCQATGYPL
AEVSWPNVSVPANTSHSRTPEGLYQVTSVLRLKPPPGRNFSCVFWNTHV
RELTLASIDLQSQMEPRTHPTWEHIFIPFCITAFIFIATVIALRKQLCQ
KLYSSKDTTKRPVTTTKREVNSAI

Agents directed to PD-1 in any of the aspects or embodiments of the present invention include a monoclonal antibody (mAb), or antigen binding fragment thereof, which specifically binds to PD-1. In some embodiments, the mAb to PD-1 specifically binds to human PD-1. The mAb may be a human antibody, a humanized antibody or a chimeric antibody, and may include a human constant region. The human constant region is selected from the group consisting of IgG1, IgG2, IgG3 and IgG4 constant regions, and in preferred embodiments, the human constant region is an IgG1 or IgG4 constant region. The antigen binding fragment may be selected from the group consisting of Fab, Fab′-SH, F(ab′)2, scFv and Fv fragments.

Examples of mAbs that bind to human PD-1 are described in U.S. Pat. Nos. 8,552,154; 8,354,509; 8,168,757; 8,008,449; 7,521,051; 7,488,802; WO2004072286; WO2004056875; and WO2004004771.

Other PD-1 binding proteins include an immunoadhesin that specifically binds to PD-1, and preferably specifically binds to human PD-1, e.g., a fusion protein containing the extracellular or PD-1 binding portion of PD-L1 or PD-L2 fused to a constant region such as an Fc region of an immunoglobulin molecule. Examples of immunoadhesin molecules that specifically bind to PD-1 are described in WO2010027827 and WO2011066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

OPDIVO/nivolumab is a fully human monoclonal antibody marketed by Bristol Myers Squibb directed against the negative immunoregulatory human cell surface receptor PD-1 (programmed death-1 or programmed cell death-1/PCD-1) with immunopotentiation activity. Nivolumab binds to and blocks the activation of PD-1, an Ig superfamily transmembrane protein, by its ligands PD-L1 and PD-L2, resulting in the activation of T-cells and cell-mediated immune responses against tumor cells or pathogens. Activated PD-1 negatively regulates T-cell activation and effector function through the suppression of P13k/Akt pathway activation. Other names for nivolumab include: BMS-936558, MDX-1106, and ONO-4538. The amino acid sequence for nivolumab and methods of using and making are disclosed in U.S. Pat. No. 8,008,449.

KEYTRUDA/pembrolizumab is an anti-PD-1 antibody marketed for the treatment of lung cancer by Merck. The amino acid sequence of pembrolizumab and methods of using are disclosed in U.S. Pat. No. 8,168,757.

LIBTAYO/cemiplimab-rwlc is an anti-PD-1 antibody marketed by Regeneron and Sanofi for treatment of advanced cutaneous squamous cell carcinoma.

By “agent directed to PD-L1” is meant any chemical compound or biological molecule capable of binding to PD-L1. In some embodiments, the agent directed to PD-L1 is a PD-L1 binding protein. The term “PDL1 binding protein” or “PD-L1 binding protein” as used herein refers to antibodies and other protein constructs, such as domains, which are capable of binding to PD-L1. In some instances, the PD-L1 is human PD1. The term “PD-L1 binding protein” can be used interchangeably with “PD-L1 antigen binding protein.” Thus, as is understood in the art, anti-PD-L1 antibodies and/or PD-L1 antigen binding proteins would be considered PD-L1 binding proteins. As used herein, “antigen binding protein” is any protein, including but not limited to antibodies, domains and other constructs described herein, that binds to an antigen, such as PD-L1. As used herein “antigen binding portion” of a PD-L1 binding protein would include any portion of the PD-L1 binding protein capable of binding to PD-L1, including but not limited to, an antigen binding antibody fragment.

The agent directed to PD-L1 may be a PD1 antagonist and blocks binding of PD-L1 expressed on a cancer cell to PD-1 expressed on an immune cell (T cell, B cell or NKT cell) and may also block binding of PD-L2 expressed on a cancer cell to the immune-cell expressed PD-1.

PD-L1 is a B7 family member that is expressed on many cell types, including APCs and activated T cells (Yamazaki et al. (2002) J. Immunol. 169:5538). PD-L1 binds to both PD-1 and B7-1. Both binding of T-cell-expressed B7-1 by PD-L1 and binding of T-cell-expressed PD-L1 by B7-1 result in T cell inhibition (Butte et al. (2007) Immunity 27:111). There is also evidence that, like other B7 family members, PD-L1 can also provide costimulatory signals to T cells (Subudhi et al. (2004) J. Clin. Invest. 113:694; Tamura et al. (2001) Blood 97:1809). PD-L1 (human PD-L1 cDNA is composed of the base sequence shown by EMBL/GenBank Acc. No. AF233516 and mouse PD-L1 cDNA is composed of the base sequence shown by NM.sub.--021893) that is a ligand of PD-1 is expressed in so-called antigen-presenting cells (APCs) such as activated monocytes and dendritic cells (Journal of Experimental Medicine (2000), vol. 19, issue 7, p 1027-1034). These cells present interaction molecules that induce a variety of immuno-inductive signals to T lymphocytes, and PD-L1 is one of these molecules that induce the inhibitory signal by PD-1. It has been revealed that PD-L1 ligand stimulation suppressed the activation (cellular proliferation and induction of various cytokine production) of PD-1 expressing T lymphocytes. PD-L1 expression has been confirmed in not only immunocompetent cells but also a certain kind of tumor cell lines (cell lines derived from monocytic leukemia, cell lines derived from mast cells, cell lines derived from hepatic carcinomas, cell lines derived from neuroblasts, and cell lines derived from breast carcinomas) (Nature Immunology (2001), vol. 2, issue 3, p. 261-267).

Anti-PD-L1 antibodies and methods of making the same are known in the art. Such antibodies to PD-L1 may be polyclonal or monoclonal, and/or recombinant, and/or humanized, and/or fully human. PD-L1 antibodies are in development as immuno-modulatory agents for the treatment of cancer.

PD-L1 antibodies are disclosed in U.S. Pat. Nos. 9,212,224; 8,779,108; 8,552,154; 8,383,796; 8,217,149; US Patent Publication No. 20110280877; WO2013079174; and WO2013019906. Additional exemplary antibodies to PD-L1 (also referred to as CD274 or B7-H1) and methods for use are disclosed in U.S. Pat. Nos. 8,168,179; 7,943,743; 7,595,048; WO2014055897; WO2013019906; and WO2010077634. Specific anti-human PD-L1 monoclonal antibodies useful as a PD-1 antagonist in the treatment method, medicaments and uses of the present invention include MPDL3280A, BMS-936559, MEDI4736, MSB0010718C.

Atezolizumab is a fully humanized monoclonal anti-PD-L1 antibody commercially available as TECENTRIQ. Atezolizumab is indicated for the treatment of some locally advanced or metastatic urothelial carcinomas. Atezolizumab blocks the interaction of PD-L1 with PD-1 and CD80. Avelumab is an anti-PD-L1 antibody commercially available as BAVENCIO.

Durvalumab (previously known as MEDI4736) is a human monoclonal antibody directed against PD-L1. Durvalumab blocks the interaction of PD-L1 with PD-1 and CD80. Durvalumab is commercially available as IMFINZI™.

Antibodies to PD-L1 (also referred to as CD274 or B7-H1) and methods for use are disclosed in U.S. Pat. Nos. 7,943,743; 8,383,796; US20130034559, WO2014055897, U.S. Pat. Nos. 8,168,179; and 7,595,048. PD-L1 antibodies are in development as immuno-modulatory agents for the treatment of cancer.

As used herein an “immuno-modulator” or “immuno-modulatory agent” refers to any substance including monoclonal antibodies that affects the immune system. In some embodiments, the immuno-modulator or immuno-modulatory agent upregulates the immune system. Immuno-modulators can be used as anti-neoplastic agents for the treatment of cancer. For example, immuno-modulators include, but are not limited to, anti-PD-1 antibodies (Opdivo/nivolumab and Keytruda/pembrolizumab), anti-CTLA-4 antibodies such as ipilimumab (YERVOY), and anti-ICOS antibodies.

As used herein the term “agonist” refers to an antigen binding protein including but not limited to an antibody, which upon contact with a co-signalling receptor causes one or more of the following (1) stimulates or activates the receptor, (2) enhances, increases or promotes, induces or prolongs an activity, function or presence of the receptor and/or (3) enhances, increases, promotes or induces the expression of the receptor. Agonist activity can be measured in vitro by various assays know in the art such as, but not limited to, measurement of cell signalling, cell proliferation, immune cell activation markers, cytokine production. Agonist activity can also be measured in vivo by various assays that measure surrogate end points such as, but not limited to the measurement of T cell proliferation or cytokine production.

As used herein the term “antagonist” refers to an antigen binding protein including but not limited to an antibody, which upon contact with a co-signalling receptor causes one or more of the following (1) attenuates, blocks or inactivates the receptor and/or blocks activation of a receptor by its natural ligand, (2) reduces, decreases or shortens the activity, function or presence of the receptor and/or (3) reduces, decrease, abrogates the expression of the receptor. Antagonist activity can be measured in vitro by various assays know in the art such as, but not limited to, measurement of an increase or decrease in cell signalling, cell proliferation, immune cell activation markers, cytokine production. Antagonist activity can also be measured in vivo by various assays that measure surrogate end points such as, but not limited to the measurement of T cell proliferation or cytokine production.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanized, multispecific antibodies, including bispecific antibodies, and heteroconjugate antibodies; a single variable domain (e.g., V_{H}, V_{HH}, VL, domain antibody (dAb™)), antigen binding antibody fragments, Fab, F(ab)2, Fv, disulphide linked Fv, single chain Fv, disulphide-linked scFv, diabodies, TANDABS™, etc. and modified versions of any of the foregoing (for a summary of alternative “antibody” formats see, e.g., Holliger and Hudson, Nature Biotechnology, 2005, Vol 23, No. 9, 1126-1136).

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain.

The term “domain” refers to a folded protein structure which retains its tertiary structure independent of the rest of the protein. Generally domains are responsible for discrete functional properties of proteins and in many cases may be added, removed or transferred to other proteins without loss of function of the remainder of the protein and/or of the domain.

The term “single variable domain” refers to a folded polypeptide domain comprising sequences characteristic of antibody variable domains. It therefore includes complete antibody variable domains such as V_{H}, V_{HH} and V_{L} and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen or epitope independently of a different variable region or domain. A “domain antibody” or “dAb™” may be considered the same as a “single variable domain”. A single variable domain may be a human single variable domain, but also includes single variable domains from other species such as rodent nurse shark and Camelid V_{HH} dAbs™. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanized according to standard techniques available in the art, and such domains are considered to be “single variable domains”. As used herein V_{H} includes camelid V_{HH} domains.

An antigen binding fragment may be provided by means of arrangement of one or more CDRs on non-antibody protein scaffolds. “Protein Scaffold” as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions.

The protein scaffold may be an Ig scaffold, for example an IgG, or IgA scaffold. The IgG scaffold may comprise some or all the domains of an antibody (i.e. CH1, CH2, CH3, V_{H}, V_{L}). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof.

Affinity is the strength of binding of one molecule, e.g. an antigen binding protein of the invention, to another, e.g. its target antigen, at a single binding site. The binding affinity of an antigen binding protein to its target may be determined by equilibrium methods (e.g. enzyme-linked immunoabsorbent assay (ELISA) or radioimmunoassay (RIA)), or kinetics (e.g. BIACORE™ analysis). Avidity is the sum total of the strength of binding of two molecules to one another at multiple sites, e.g. taking into account the valency of the interaction.

By “isolated” it is intended that the molecule, such as an antigen binding protein or nucleic acid, is removed from the environment in which it may be found in nature. For example, the molecule may be purified away from substances with which it would normally exist in nature. For example, the mass of the molecule in a sample may be 95% of the total mass.

The term “expression vector” as used herein means an isolated nucleic acid which can be used to introduce a nucleic acid of interest into a cell, such as a eukaryotic cell or prokaryotic cell, or a cell free expression system where the nucleic acid sequence of interest is expressed as a peptide chain such as a protein. Such expression vectors may be, for example, cosmids, plasmids, viral sequences, transposons, and linear nucleic acids comprising a nucleic acid of interest. Once the expression vector is introduced into a cell or cell free expression system (e.g., reticulocyte lysate) the protein encoded by the nucleic acid of interest is produced by the transcription/translation machinery. Expression vectors within the scope of the disclosure may provide necessary elements for eukaryotic or prokaryotic expression and include viral promoter driven vectors, such as CMV promoter driven vectors, e.g., pcDNA3.1, pCEP4, and their derivatives, Baculovirus expression vectors, Drosophila expression vectors, and expression vectors that are driven by mammalian gene promoters, such as human Ig gene promoters. Other examples include prokaryotic expression vectors, such as T7 promoter driven vectors, e.g., pET41, lactose promoter driven vectors and arabinose gene promoter driven vectors. Those of ordinary skill in the art will recognize many other suitable expression vectors and expression systems.

The term “recombinant host cell” as used herein means a cell that comprises a nucleic acid sequence of interest that was isolated prior to its introduction into the cell. For example, the nucleic acid sequence of interest may be in an expression vector while the cell may be prokaryotic or eukaryotic. Exemplary eukaryotic cells are mammalian cells, such as but not limited to, COS-1, COS-7, HEK293, BHK21, CHO, BSC-1, HepG2, 653, SP2/0, NS0, 293, HeLa, myeloma, lymphoma cells or any derivative thereof. Most preferably, the eukaryotic cell is a HEK293, NS0, SP2/0, or CHO cell. E. coli is an exemplary prokaryotic cell. A recombinant cell according to the disclosure may be generated by transfection, cell fusion, immortalization, or other procedures well known in the art. A nucleic acid sequence of interest, such as an expression vector, transfected into a cell may be extrachromosomal or stably integrated into the chromosome of the cell.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one or more human immunoglobulin(s). In addition, framework support residues may be altered to preserve binding affinity (see, e.g., Queen et al. Proc. Natl Acad Sci USA, 86:10029-10032 (1989), Hodgson, et al., Bio/Technology, 9:421 (1991)). A suitable human acceptor antibody may be one selected from a conventional database, e.g., the KABAT™ database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies—see, for example, EP-A-0239400 and EP-A-054951.

The term “fully human antibody” includes antibodies having variable and constant regions (if present) derived from human germline immunoglobulin sequences. The human sequence antibodies of the invention may include amino acid residues not encoded by human germline immunoglobulin sequences (e.g., mutations introduced by random or site-specific mutagenesis in vitro or by somatic mutation in vivo). Fully human antibodies comprise amino acid sequences encoded only by polynucleotides that are ultimately of human origin or amino acid sequences that are identical to such sequences. As meant herein, antibodies encoded by human immunoglobulin-encoding DNA inserted into a mouse genome produced in a transgenic mouse are fully human antibodies since they are encoded by DNA that is ultimately of human origin. In this situation, human immunoglobulin-encoding DNA can be rearranged (to encode an antibody) within the mouse, and somatic mutations may also occur. Antibodies encoded by originally human DNA that has undergone such changes in a mouse are fully human antibodies as meant herein. The use of such transgenic mice makes it possible to select fully human antibodies against a human antigen. As is understood in the art, fully human antibodies can be made using phage display technology wherein a human DNA library is inserted in phage for generation of antibodies comprising human germline DNA sequence.

The term “donor antibody” refers to an antibody that contributes the amino acid sequences of its variable regions, CDRs, or other functional fragments or analogs thereof to a first immunoglobulin partner. The donor, therefore, provides the altered immunoglobulin coding region and resulting expressed altered antibody with the antigenic specificity and neutralising activity characteristic of the donor antibody.

The term “acceptor antibody” refers to an antibody that is heterologous to the donor antibody, which contributes all (or any portion) of the amino acid sequences encoding its heavy and/or light chain framework regions and/or its heavy and/or light chain constant regions to the first immunoglobulin partner. A human antibody may be the acceptor antibody.

The terms “V_{H}” and “V_{L}” are used herein to refer to the heavy chain variable region and light chain variable region respectively of an antigen binding protein.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and full length antibody sequences are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 5th Ed., U.S. Department of Health and Human Services, National Institutes of Health (1991).

It will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

“Percent identity” between a query amino acid sequence and a subject amino acid sequence is the “Identities” value, expressed as a percentage, that is calculated using a suitable algorithm or software, such as BLASTP, FASTA, DNASTAR Lasergene, GeneDoc, Bioedit, EMBOSS needle or EMBOSS infoalign, over the entire length of the query sequence after a pair-wise global sequence alignment has been performed using a suitable algorithm/software such as BLASTP, FASTA, ClustalW, MUSCLE, MAFFT, EMBOSS Needle, T-Coffee, and DNASTAR Lasergene. Importantly, a query amino acid sequence may be described by an amino acid sequence identified in one or more claims herein.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The % identity may be determined across the entire length of the query sequence, including the CDRs. Alternatively, the % identity may exclude one or more or all of the CDRs, for example all of the CDRs are 100% identical to the subject sequence and the % identity variation is in the remaining portion of the query sequence, e.g. the framework sequence, so that the CDR sequences are fixed and intact.

The variant sequence substantially retains the biological characteristics of the unmodified protein, such as an agonist for ICOS.

### Methods of Treatment

In one aspect, there is provided a method of treating cancer, the method comprising administering to a human an ICOS binding protein or antigen binding portion thereof at a dose of about 0.08 mg to about 240 mg. In one embodiment, the ICOS binding protein or antigen binding portion is an ICOS agonist. In still another embodiment, the method of treating cancer comprises treating cancer in a human in need thereof. In another embodiment, the ICOS binding protein or antigen binding portion thereof is administered at a dose of 8 mg, 24 mg, or 80 mg.

In one aspect, there is provided a method of treating a human with cancer, the method comprising administering to the human an agonist ICOS binding protein or antigen binding portion thereof at a dose of about 0.08 mg to about 240 mg.

In one aspect, there is provided a method of treating cancer in a human in need thereof, the method comprising administering to the human an agonist ICOS binding protein or antigen binding portion thereof at a dose of about 0.08 mg to about 240 mg. In one embodiment, the ICOS binding protein or antigen binding portion thereof is administered at a dose of 8 mg, 24 mg, or 80 mg.

In another aspect, an agonist ICOS binding protein or antigen binding portion thereof for use in treating cancer is provided, wherein the ICOS binding protein or antigen binding portion thereof is to be administered at a dose of about 0.08 mg to about 240 mg. In one embodiment, the ICOS binding protein or antigen binding portion thereof is administered at a dose of 8 mg, 24 mg, or 80 mg.

In one aspect, use of an agonist ICOS binding protein or antigen binding portion thereof in the manufacture of a medicament for treating cancer is provided, wherein the agonist ICOS binding protein or antigen binding portion thereof is to be administered at a dose of about 0.08 mg to about 240 mg. In one embodiment, the ICOS binding protein or antigen binding portion thereof is administered at a dose of 8 mg, 24 mg, or 80 mg.

In another aspect, a pharmaceutical kit comprising about 0.08 mg to about 240 mg of an ICOS binding protein or antigen binding portion thereof is provided. In one embodiment, the ICOS binding protein or antigen binding portion thereof is administered at a dose of 8 mg, 24 mg, or 80 mg. In one embodiment, the pharmaceutical kit comprises the ICOS binding protein or antigen binding portion thereof at a concentration of 10 mg/ml.

In another aspect, there is provided a pharmaceutical formulation comprising an ICOS binding protein or antigen binding portion thereof at a concentration of 10 mg/ml.

The individual components of the combinations disclosed herein may be administered either in separate or combined pharmaceutical formulations by any convenient route.

In one embodiment, the dose of the ICOS binding protein or antigen binding portion thereof is in the range of about 0.08 mg to about 800 mg. In another embodiment, the dose of the ICOS binding protein or antigen binding portion thereof is in the range of about 0.8 mg to about 240 mg. In another embodiment, the dose of the ICOS binding protein or antigen binding portion thereof is in the range of about 8 mg to about 80 mg. In another embodiment, the dose of the ICOS binding protein or antigen binding portion thereof is about 0.08 mg, about 0.24 mg, about 0.8 mg, about 2.4 mg, about 8 mg, about 24 mg, about 80 mg, or about 240 mg. In one embodiment, the dose of ICOS binding protein or antigen binding portion thereof is about 8 mg, about 24 mg, or about 80 mg. In one embodiment, the dose of ICOS binding protein or antigen binding portion thereof is at least 240 mg. In one embodiment, the dose of the agonist ICOS binding protein or antigen binding portion thereof is at least 80 mg.

It is to be understood that where mg/kg is used, this is mg/kg of body weight. In one embodiment, the dose of the ICOS binding protein or antigen binding portion thereof is between about 0.001 mg/kg to about 3.0 mg/kg. In another embodiment, the dose of the ICOS binding protein or antigen binding portion thereof is about 0.001 mg/kg, about 0.003 mg/kg, about 0.01 mg/kg, about 0.03 mg/kg, about 0.1 mg/kg, about 0.3 mg/kg, about 1.0 mg/kg, about 3.0 mg/kg, or about 10 mg/kg. In another embodiment, the dose of the ICOS binding protein or antigen binding portion thereof is at least 3.0 mg/kg. In one embodiment, the dose of the ICOS binding protein or antigen binding portion thereof is in the range of about 0.001 mg/kg to about 10 mg/kg. In one embodiment, the dose of the ICOS binding protein or antigen binding portion thereof is about 0.1 mg/kg to about 1.0 mg/kg. In one embodiment, the dose of the ICOS binding protein or antigen binding portion thereof is about 0.1 mg/kg. In one embodiment, the dose of the ICOS binding protein or antigen binding portion thereof is at least 0.1 mg/kg. In another embodiment, the dose of the ICOS binding protein is about 0.3 mg/kg. In another embodiment, the dose of the ICOS binding protein is about 1 mg/kg. In one embodiment, the dose of the ICOS binding protein is about 3 mg/kg. In one embodiment a fixed dose of ICOS binding protein or antigen binding portion thereof may be administered, assuming a typical median weight of 80 kg.

In one embodiment, the dose of ICOS binding protein or antigen binding portion thereof is increased during the treatment regimen. In one embodiment an initial dose of about 0.001 mg/kg, about 0.003 mg/kg, about 0.01 mg/kg, about 0.03 mg/kg, about 0.1 mg/kg, about 0.3 mg/kg, about 1.0 mg/kg is increased to about 0.003 mg/kg, about 0.01 mg/kg, about 0.03 mg/kg, about 0.1 mg/kg, about 0.3 mg/kg, about 1.0 mg/kg, about 3.0 mg/kg or at least 3.0 mg/kg. In one embodiment, an initial dose of 0.1 mg/kg is increased to 1 mg/kg. In one embodiment, an initial dose of 0.3 mg/kg is increased to 1 mg/kg.

In one embodiment, the ICOS binding protein or antigen binding portion thereof is administered at 0.1 mg/kg×3 doses then 1 mg/kg. In one embodiment, the ICOS binding protein or antigen binding portion thereof is administered at about 0.001 mg/kg, about 0.003 mg/kg, about 0.01 mg/kg, about 0.03 mg/kg, about 0.1 mg/kg, about 0.3 mg/kg, about 1.0 mg/kg, or about 3.0 mg/kg then increased to about 0.01 mg/kg, about 0.03 mg/kg, about 0.1 mg/kg, about 0.3 mg/kg, about 1.0 mg/kg, about 3.0 mg/kg or about 10 mg/kg.

Fixed doses may be tested assuming a typical median weight of 80 kg.

Therapeutic monoclonal antibodies are often dosed based on body-size due to the concept that this reduces inter-subject variability in drug exposure. However, body-weight dependency of PK parameters does not always explain the observed variability in the exposure of monoclonal antibodies (Zhao X, Suryawanshi, S; Hruska, M. Assessment of nivolumab benefit-risk profile of a 240-mg flat dose relative to a 3 mg/kg dosing regimen in patient with advanced tumors. Annals of Oncology. 2017; 28:2002-2008). The advantage of body-weight based versus fixed dosing in the study provide in the Examples was evaluated through population PK modelling and simulation efforts. A preliminary population PK model was developed from monotherapy dose escalation (data up to doses of 1 mg/kg; n=19 subjects).

Simulations were performed by considering body weight distribution in the simulations were based on the observed distribution in the preliminary dataset. At the 5th percentile of body weight (40-47 kg), there was a 70-100% increase in median steady-state AUC(0-); H2L5 IgG4PE exposures higher than these increases have been evaluated in the current Phase 1 study with the 3 mg/kg dose regimen. At the 95th percentile of body weight (107-118 kg), there was a 23-32% decrease in median steady-state AUC (0-) as compared to the median 80 kg exposure providing adequate receptor occupancy (RO) with the minimal lowering of exposure. A similar outcome is expected for steady-state Cmax and trough concentrations between body weight-based and fixed dosing.

Overall, these preliminary population PK simulations indicate that using fixed dosing would result in a similar range of exposures as that of body weight-based dosing. Also, fixed dosing offers the advantage of reduced dosing errors, reduced drug wastage, shorten preparation time, and improve ease of administration. Thus, switching to a fixed dose based on a reference body weight of 80 kg is reasonable and appropriate.

The fixed dose equivalents of the weight-based H2L5 IgG4PE dose levels using 80 kg weight are presented in Table 3.

In one embodiment, the ICOS binding protein or antigen binding portion thereof is administered via IV infusion. In one embodiment, the PD1 antagonist is administered via IV infusion. In one embodiment, the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the PD1 antagonist is pembrolizumab.

In one embodiment, the ICOS binding protein or antigen binding portion thereof is administered once every 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, or 40 days.

In one embodiment, the ICOS binding protein or antigen binding portion thereof is administered once every week, once every two weeks, once every three weeks, or once every four weeks. In one embodiment, the ICOS binding protein or antigen binding portion thereof is administered once every three weeks. In one embodiment, the PD1 antagonist is administered once every three weeks. In one embodiment, the ICOS binding protein or antigen binding portion thereof is administered once every three weeks until disease progression. In one embodiment, the ICOS binding protein or antigen binding portion thereof is administered once every three weeks for 35 cycles. In one embodiment, the ICOS binding protein or antigen binding portion thereof is administered via IV infusion at a dose of about 0.08 mg, about 0.24 mg, about 0.8 mg, about 2.4 mg, about 8 mg, about 24 mg, about 80 mg, or about 240 mg every three weeks. In one embodiment, the ICOS binding protein or antigen thereof is administered at a dose of 24 mg via IV infusion every three weeks. In one embodiment, the ICOS binding protein or antigen binding portion thereof and/or PD1 antagonist is administered every three weeks until disease progression. In one embodiment, the ICOS binding protein or antigen binding portion thereof and/or PD1 antagonist is administered every three weeks up to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 cycles. In one embodiment, the ICOS binding protein or antigen binding portion thereof and/or PD1 antagonist is administered every three weeks up to 35 cycles.

In some embodiments, the patient is first administered the ICOS binding protein or antigen binding portion thereof as a monotherapy regimen and then the ICOS binding protein or antigen binding portion thereof with the PD1 antagonist as a combination therapy regimen. In some embodiments, the patient is administered the ICOS binding protein or antigen binding portion thereof as a monotherapy regimen.

In one aspect, there is provided a method of treating cancer in a human in need thereof, the method comprising administering to the human an ICOS binding protein or antigen binding portion thereof at a dose of about 8 mg to about 80 mg, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein or antigen binding portion thereof is administered at a dose of 8 mg, 24 mg, or 80 mg. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a VH domain comprising the amino acid sequence set forth in SEQ ID NO:7 and a VL domain comprising the amino acid sequence as set forth in SEQ ID NO:8. In one embodiment, the ICOS binding protein comprises a heavy chain comprising the amino acid sequence set forth in SEQ ID NO: 34 and a light chains comprising the amino acid sequence as set forth in SEQ ID NO: 35.

In another aspect, an agonist ICOS binding protein or antigen binding portion thereof for use in treating cancer is provided, wherein the ICOS binding protein or antigen binding portion thereof is to be administered at a dose of about 8 mg to about 80 mg, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein or antigen binding portion thereof is administered at a dose of 8 mg, 24 mg, or 80 mg. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a VH domain comprising the amino acid sequence set forth in SEQ ID NO:7 and a VL domain comprising the amino acid sequence as set forth in SEQ ID NO:8. In one embodiment, the ICOS binding protein comprises a heavy chain comprising the amino acid sequence set forth in SEQ ID NO: 34 and a light chains comprising the amino acid sequence as set forth in SEQ ID NO: 35. In another aspect, there is provided use of an agonist ICOS binding protein or antigen binding portion thereof in the manufacture of a medicament for treating cancer, wherein the agonist ICOS binding protein or antigen binding portion thereof is to be administered at a dose of about 8 mg to about 80 mg, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein or antigen binding portion thereof is administered at a dose of 8 mg, 24 mg, or 80 mg. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a VH domain comprising the amino acid sequence set forth in SEQ ID NO:7 and a VL domain comprising the amino acid sequence as set forth in SEQ ID NO:8. In one embodiment, the ICOS binding protein comprises a heavy chain comprising the amino acid sequence set forth in SEQ ID NO: 34 and a light chains comprising the amino acid sequence as set forth in SEQ ID NO: 35.

In still another aspect, there is provided a pharmaceutical kit comprising about 8 mg to about 80 mg an ICOS binding protein or antigen binding portion thereof, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the kit comprises 8 mg, 24 mg, or 80 mg of an ICOS binding protein or antigen binding portion thereof. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a VH domain comprising the amino acid sequence set forth in SEQ ID NO:7 and a VL domain comprising the amino acid sequence as set forth in SEQ ID NO:8. In one embodiment, the ICOS binding protein comprises a heavy chain comprising the amino acid sequence set forth in SEQ ID NO: 34 and a light chains comprising the amino acid sequence as set forth in SEQ ID NO: 35.

In one aspect, there is provided a method of treating cancer, the method comprising administering to a human an agonist ICOS binding protein or an antigen binding fragment thereof at a dose wherein the median plasma concentration of the agonist ICOS binding protein is between 100 μg/ml and 0.1 μg/ml for at least 7 days after the first dose.

In one aspect, there is provided an agonist ICOS binding protein or an antigen binding fragment thereof for use in the treatment of cancer, wherein agonist ICOS binding protein or an antigen binding fragment thereof is administered at a dose wherein the median plasma concentration of the agonist ICOS binding protein is between 100 μg/ml and 0.1 μg/ml for at least 7 days after the first dose.

In another aspect, there is provided use of an agonist ICOS binding protein or an antigen binding fragment thereof in the manufacture of a medicament for treating cancer, wherein the agonist ICOS binding protein or an antigen binding fragment thereof is administered at a dose wherein the median plasma concentration of the agonist ICOS binding protein is between 100 μg/ml and 0.1 μg/ml for at least 7 days after the first dose.

In one embodiment, the agonist ICOS binding protein or an antigen binding fragment thereof is administered at a dose wherein the median plasma concentration of the agonist ICOS binding protein is between 100 μg/ml, 10 μg/ml, 1 μg/ml or 0.1 μg/ml and 10 μg/ml, 1 μg/ml or 0.1 μg/ml for at least 1, 2.5, 4.5, 7, 14 or 21 days after the first dose.

In one embodiment, the agonist ICOS binding protein or an antigen binding fragment thereof is administered at a dose wherein the median plasma concentration of the agonist ICOS binding protein is between 100 μg/ml, 90 μg/ml, 80 μg/ml, 70 μg/ml, 60 μg/ml, 50 μg/ml, 40 μg/ml, 30 μg/ml, 20 μg/ml, 10 μg/ml, 9 μg/ml, 8 μg/ml, 7 μg/ml, 6 μg/ml, 5 μg/ml, 4 μg/ml, 3 μg/ml, 2 μg/ml, 1 μg/ml, 0.9 μg/ml, 0.8 μg/ml, 0.7 μg/ml, 0.6 μg/ml, 0.5 μg/ml, 0.4 μg/ml, 0.3 μg/ml or 0.2 μg/ml and 90 μg/ml, 80 μg/ml, 70 μg/ml, 60 μg/ml, 50 μg/ml, 40 μg/ml, 30 μg/ml, 20 μg/ml, 10 μg/ml, 9 μg/ml, 8 μg/ml, 7 μg/ml, 6 μg/ml, 5 μg/ml, 4 μg/ml, 3 μg/ml, 2 μg/ml, 1 μg/ml, 0.9 μg/ml, 0.8 μg/ml, 0.7 μg/ml, 0.6 μg/ml, 0.5 μg/ml, 0.4 μg/ml, 0.3 μg/ml, 0.2 μg/ml or 0.1 10 μg/ml, for at least 1, 2, 2.5, 3, 4, 4.5, 5, 6, 7, 8, 9, 10, 11, 12, 13, 14, 15, 16, 17, 18, 19, 20 or 21 days after the first dose.

In one embodiment, the human is administered an agonist ICOS binding protein or an antigen binding fragment thereof at a dose wherein the median plasma concentration of the agonist ICOS binding protein is between 10 μg/ml and 1 μg/ml at 21 days after first dose. In one embodiment, the human is administered an agonist ICOS binding protein or an antigen binding fragment thereof at a dose wherein the median plasma concentration of the agonist ICOS binding protein is between 10 μg/ml and 0.1 μg/ml at 21 days after first dose.

In one embodiment, the human is administered an agonist ICOS binding protein or an antigen binding fragment thereof at a dose wherein the median plasma concentration of the agonist ICOS binding protein is between 100 μg/ml and 1 μg/ml at 21 days after first dose. In one embodiment, the human is administered an agonist ICOS binding protein or an antigen binding fragment thereof at a dose wherein the median plasma concentration of the agonist ICOS binding protein is between 100 μg/ml and 10 μg/ml at 21 days after first dose.

In one aspect, there is provided a method of treating cancer, the method comprising administering to a human an agonist ICOS binding protein or an antigen binding fragment thereof at a dose wherein ICOS receptor saturation in the human is at or above around 50% for at least 7 days after first dose.

In one aspect, there is provided an agonist ICOS binding protein or an antigen binding fragment thereof for use in the treatment of cancer, wherein agonist ICOS binding protein or an antigen binding fragment thereof is administered to a human at a dose wherein ICOS receptor saturation in the human is at or above around 50% for at least 7 days after first dose.

In another aspect, there is provided use of an agonist ICOS binding protein or an antigen binding fragment thereof in the manufacture of a medicament for treating cancer, wherein the agonist ICOS binding protein or an antigen binding fragment thereof is administered to a human at a dose wherein ICOS receptor saturation in the human is at or above around 50% for at least 7 days after first dose.

In one embodiment, the human is administered an agonist ICOS binding protein or an antigen binding fragment thereof at a dose wherein ICOS receptor saturation (i.e. receptor occupancy) in the human is at or above around 50%, 55%, 60%, 65%, 70%, 75%, 80%, 85%, 90% or 95% for at least 1, 2, 3, 4, 5, 6, 7, 8, 9, 10, 11, 12, 13, 14, 15, 16, 17, 18, 19, 20 or 21 days after first dose.

In one aspect, there is provided a method of treating cancer, the method comprising administering to a human an agonist ICOS binding protein or an antigen binding fragment thereof at a dose peripheral CD4⁺ or CD8⁺ T cell receptor occupancy is at or above 50% for at least 7 days after first dose.

In one aspect, there is provided an agonist ICOS binding protein or an antigen binding fragment thereof for use in the treatment of cancer, wherein agonist ICOS binding protein or an antigen binding fragment thereof is administered to a human at a dose peripheral CD4⁺ or CD8⁺ T cell receptor occupancy is at or above 50% for at least 7 days after first dose.

In another aspect, there is provided use of an agonist ICOS binding protein or an antigen binding fragment thereof in the manufacture of a medicament for treating cancer, wherein the agonist ICOS binding protein or an antigen binding fragment thereof is administered to a human at a dose peripheral CD4⁺ or CD8⁺ T cell receptor occupancy is at or above 50% for at least 7 days after first dose.

Peak CD4⁺ Receptor Occupancy (RO) corresponds to agonist ICOS binding protein or antigen fragment thereof maximum plasma concentration. Peak CD8⁺ Receptor Occupancy (RO) corresponds to agonist ICOS binding protein or antigen fragment thereof maximum plasma concentration.

In one embodiment, the agonist ICOS binding protein or an antigen binding fragment thereof is administered at a dose wherein peripheral CD4⁺ or CD8⁺ T cell receptor occupancy is at or above around 50%, 55%, 60%, 65%, 70%, 75%, 80%, 85%, 90% or 95% for at least 1, 2, 3, 4, 5, 6, 7, 8, 9, 10, 11, 12, 13, 14, 15, 16, 17, 18, 19, 20 or 21 days after first dose.

In one embodiment, the agonist ICOS binding protein or an antigen binding fragment thereof is administered at a dose wherein peripheral CD4⁺ or CD8⁺ T cell receptor occupancy is at or above around 60%, for at least 21 days after first dose. In one embodiment, the agonist ICOS binding protein or an antigen binding fragment thereof is administered at a dose wherein peripheral CD4⁺ or CD8⁺ T cell receptor occupancy is at or above around 70%, for at least 21 days after first dose. In one embodiment, the agonist ICOS binding protein or an antigen binding fragment thereof is administered at a dose wherein peripheral CD4⁺ or CD8⁺ T cell receptor occupancy is at or above around 80%, for at least 21 days after first dose. In one embodiment, the agonist ICOS binding protein or an antigen binding fragment thereof is administered at a dose wherein peripheral CD4⁺ or CD8⁺ T cell receptor occupancy is at or above around 90%, for at least 21 days after first dose.

In one aspect, there is provided a pharmaceutical composition comprising an agonist ICOS binding protein or an antigen binding fragment thereof, wherein said composition provides an Area Under the Curve value of 37 mg/mL×day to 255 mg/mL×day of the agonist ICOS binding protein or the antigen binding fragment thereof after a single dose. In one embodiment, said composition further provides a PD1 antagonist. In one embodiment, said composition further provides a PD1 antagonist. In one embodiment, said composition provides an AUC value of 62 mg/mL×day to 220 mg/mL×day of the ICOS binding protein or the antigen fragment thereof after a single dose.

In one aspect, there is provided a method of treating cancer in a human in need thereof, the method comprising administering to the human an agonist ICOS binding protein or antigen binding portion thereof. In another aspect, there is provided an agonist ICOS binding protein or antigen binding portion thereof for use in treating cancer. In a further aspect, there is provided use of an agonist ICOS binding protein or antigen binding portion thereof in the manufacture of a medicament for treating cancer. There is disclosed a pharmaceutical kit comprising an ICOS binding protein or antigen binding portion thereof.

In one aspect, there is provided a method of treating cancer in a human in need thereof, the method comprising administering to the human an agonist ICOS binding protein or antigen binding portion thereof and a PD1 antagonist. In a further aspect, there is provided an agonist ICOS binding protein or antigen binding portion thereof and a PD1 antagonist for concurrent or sequential use in treating cancer. In another aspect, there is provided an ICOS binding protein or antigen binding portion thereof for use in treating cancer is provided, wherein the ICOS binding protein or antigen binding portion thereof is to be administered concurrently or sequentially with a PD1 antagonist. In one aspect, there is provided the use of an agonist ICOS binding protein or antigen binding portion thereof in the manufacture of a medicament for treating cancer, wherein the agonist ICOS binding protein or antigen binding portion thereof is to be administered concurrently or sequentially with a PD1 antagonist. In another aspect, there is provided a pharmaceutical kit comprising an ICOS binding protein or antigen binding portion thereof and a PD1 antagonist.

In one embodiment the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a VH domain comprising the amino acid sequence set forth in SEQ ID NO:7 and a VL domain comprising the amino acid sequence as set forth in SEQ ID NO:8. In one embodiment, the ICOS binding protein comprises a heavy chain comprising the amino acid sequence set forth in SEQ ID NO: 34 and a light chains comprising the amino acid sequence as set forth in SEQ ID NO: 35.

In one embodiment, the cancer is head and neck cancer. In one embodiment the cancer is head and neck squamous cell carcinoma (HNSCC). In one embodiment, the cancer is recurrent/metastatic (R/M) HNSCC. In one embodiment, the cancer is recurring/refractory (R/R) HNSCC. In one embodiment, the cancer is HPV-negative or HPV-positive HNSCC. In one embodiment, the cancer is a locally advanced HNSCC. In one embodiment, the cancer is (R/M) HNSCC in PD-L1 CPS (Combined Positive Score) positive (CPS ≥1) patients. The combined positive score is as determined by an FDA-approved test. PD-L1 CPS is the number of PD-L1 staining cells (tumor cells, lymphocytes, macrophages) divided by the total number of viable tumor cells, multiplied by 100. In one embodiment, PD-L1 CPS is determined using PharmDx 22C3 In one embodiment, the cancer is HNSCC in PD-1 antagonist/PD-L1 binding protein experienced or PD-1 antagonist/PD-L1 binding protein naïve patients.

In one embodiment, chemotherapy is further administered concurrently or sequentially with agonist ICOS binding protein or antigen binding portion thereof and/or the PD1 antagonist. In one embodiment, chemotherapy is further administered concurrently or sequentially with agonist ICOS binding protein or antigen binding portion thereof and the PD1 antagonist. In one embodiment, the chemotherapy is platinum-based chemotherapy. In one embodiment, the chemotherapy is platinum-based chemotherapy and fluorouracil. In one embodiment, the platinum-based chemotherapy is paclitaxel, docetaxel, cisplatin, carboplatin or any combination thereof. In one embodiment chemotherapy is further administered concurrently or sequentially with agonist ICOS binding protein or antigen binding portion thereof and the PD1 antagonist to PD-1 antagonist/PD-L1 binding protein naïve patients.

In one embodiment, the agonist ICOS binding protein or antigen binding portion thereof and the PD1 antagonist is administered concurrently or sequentially to PD-L1 positive patients.

In one embodiment, the agonist ICOS binding protein or antigen binding portion thereof is administered at a dose of about 0.08 mg to about 240 mg. In one embodiment, the ICOS binding protein or antigen binding portion thereof is administered at a dose of 8 mg, 24 mg, or 80 mg.

In another embodiment, the agonist ICOS binding protein or antigen binding portion thereof for use in treating cancer is provided, wherein the ICOS binding protein or antigen binding portion thereof is to be administered at a dose of about 0.08 mg to about 240 mg. In one embodiment, the ICOS binding protein or antigen binding portion thereof is administered at a dose of 8 mg, 24 mg, or 80 mg.

In one embodiment, the treatment is first-line or second line treatment of HNSCC. In one embodiment, the treatment is first-line or second line treatment of recurrent/metastatic HNSCC. In one embodiment the treatment is first line treatment of recurrent/metastatic (1L R/M) HNSCC. In one embodiment, the treatment is first line treatment of 1L R/M HNSCC in a PD-L1 CPS (combined positive score) positive (CPS ≥1) patients. In one embodiment the treatment is second line treatment of recurrent/metastatic (2L R/M) HNSCC.

In one embodiment, the treatment is first-line, second-line, third-line, fourth-line or fifth-line treatment of PD-1/PD-L1-naïve HNSCC. In one embodiment, the treatment first-line, second-line, third-line, fourth-line or fifth-line treatment of PD-1/PD-L1 experienced HNSCC.

In some embodiments, the treatment results in one or more of increased tumor infiltrating lymphocytes including cytotoxic T cells, helper T cell and NK cells, increased T cells, increased granzyme B+ cells, reduced proliferating tumour cells and increased activated T cells as compared to levels prior to treatment (e.g. baseline level). Activated T cells may be observed by greater OX40 and human leukocyte antigen DR expression. In some embodiments, treatment results in upregulation of PD1 and/or PD-L1 as compared to levels prior to treatment (e.g. baseline level).

In one embodiment, the human has a solid tumor. In one embodiment, the solid tumor is advanced solid tumor. In one embodiment, the cancer is selected from head and neck cancer, squamous cell carcinoma of the head and neck (SCCHN or HNSCC), gastric cancer, melanoma, renal cell carcinoma (RCC), esophageal cancer, non-small cell lung carcinoma, prostate cancer, colorectal cancer, ovarian cancer and pancreatic cancer. In one embodiment, the cancer is selected from the group consisting of: colorectal cancer, cervical cancer, bladder cancer, urothelial cancer, head and neck cancer, melanoma, mesothelioma, non-small cell lung carcinoma, prostate cancer, esophageal cancer, and esophageal squamous cell carcinoma. In one aspect the human has one or more of the following: SCCHN, colorectal cancer (CRC), esophageal, cervical, bladder, breast, head and neck, ovarian, melanoma, renal cell carcinoma (RCC), EC squamous cell, non-small cell lung carcinoma, mesothelioma, and prostate cancer. In another aspect the human has a liquid tumor such as diffuse large B cell lymphoma (DLBCL), multiple myeloma, chronic lyphomblastic leukemia (CLL), follicular lymphoma, acute myeloid leukemia and chronic myelogenous leukemia. In one embodiment, the cancer is recurrent/metastatic squamous cell carcinoma of the head and neck (HNSCC). In one embodiment, the cancer is a locally advanced HNSCC. In one embodiment, the cancer is R/M HNSCC. In one embodiment, the cancer is R/R HNSCC. In one embodiment the cancer is R/M HNSCC in a PD-L1 CPS (combined positive score) positive (CPS >1) patient. In one embodiment, the cancer is HNSCC in PD-1 antagonist/PD-L1 binding protein experienced or PD-1 antagonist/PD-L1 binding protein naïve patients.

In one embodiment, the cancer is head and neck cancer. In one embodiment, the cancer is HNSCC. Squamous cell carcinoma is a cancer that arises from particular cells called squamous cells. Squamous cells are found in the outer layer of skin and in the mucous membranes, which are the moist tissues that line body cavities such as the airways and intestines. Head and neck squamous cell carcinoma (HNSCC) develops in the mucous membranes of the mouth, nose, and throat. HNSCC is also known as SCCHN and squamous cell carcinoma of the head and neck.

HNSCC can occur in the mouth (oral cavity), the middle part of the throat near the mouth (oropharynx), the space behind the nose (nasal cavity and paranasal sinuses), the upper part of the throat near the nasal cavity (nasopharynx), the voicebox (larynx), or the lower part of the throat near the larynx (hypopharynx). Depending on the location, the cancer can cause abnormal patches or open sores (ulcers) in the mouth and throat, unusual bleeding or pain in the mouth, sinus congestion that does not clear, sore throat, earache, pain when swallowing or difficulty swallowing, a hoarse voice, difficulty breathing, or enlarged lymph nodes.

HNSCC can metastasize to other parts of the body, such as the lymph nodes, lungs or liver.

Tobacco use and alcohol consumption are the two most important risk factors for the development of HNSCC, and their contributions to risk are synergistic. In addition, the human papillomavirus (HPV), especially HPV-16, is now a well-established independent risk factor. Patients with HNSCC have a relatively poor prognosis. Recurrent/metastatic (R/M) HNSCC is especially challenging, regardless of human papillomavirus (HPV) status, and currently, few effective treatment options are available in the art. HPV-negative HNSCC is associated with a locoregional relapse rate of 19-35% and a distant metastatic rate of 14-22% following standard of care, compared with rates of 9-18% and 5-12%, respectively, for HPV-positive HNSCC. The median overall survival for patients with R/M disease is 10-13 months in the setting of first-line chemotherapy and 6 months in the second-line setting. The current standard of care is platinum-based doublet chemotherapy with or without cetuximab. Second-line standard of care options include cetuximab, methotrexate, and taxanes. All of these chemotherapeutic agents are associated with significant side effects, and only 10-13% of patients respond to treatment. HNSCC regressions from existing systemic therapies are transient and do not add significantly increased longevity, and virtually all patients succumb to their malignancy.

In one embodiment, the cancer is recurrent/metastatic (R/M) HNSCC. In one embodiment, the cancer is HPV-negative or HPV-positive HNSCC. In one embodiment, the cancer is a locally advanced HNSCC. In one embodiment, the cancer is R/M HNSCC in a PD-L1 CPS (combined positive score) positive (CPS ≥1) patient. In one embodiment, the cancer is HNSCC in PD-1 antagonist/PD-L1 binding protein experienced or PD-1 antagonist/PD-L1 binding protein naïve patients.

In some embodiments, the treatment of cancer is first-line treatment of cancer. In one embodiment, the treatment of cancer is second-line treatment of cancer. In some embodiments, the treatment is third-line treatment of cancer. In some embodiments, the treatment is fourth-line treatment of cancer. In some embodiments, the treatment is fifth-line treatment of cancer. In some embodiments, prior treatment to said second-line, third-line, fourth-line or fifth-line treatment of cancer comprises one or more of radiotherapy, chemotherapy, surgery or radiochemotherapy.

In one embodiment, the prior treatment comprises treatment with diterpenoids, such as paclitaxel or docetaxel; vinca alkaloids, such as vinblastine, vincristine, or vinorelbine; platinum coordination complexes, such as cisplatin or carboplatin; nitrogen mustards such as cyclophosphamide, melphalan, or chlorambucil; alkyl sulfonates such as busulfan; nitrosoureas such as carmustine; triazenes such as dacarbazine; actinomycins such as dactinomycin; anthrocyclins such as daunorubicin or doxorubicin; bleomycins; epipodophyllotoxins such as etoposide or teniposide; antimetabolite anti-neoplastic agents such as fluorouracil, methotrexate, cytarabine, mecaptopurine, thioguanine, or gemcitabine; methotrexate; camptothecins such as irinotecan or topotecan; rituximab; ofatumumab; trastuzumab; cetuximab; bexarotene; sorafenib; erbB inhibitors such as lapatinib, erlotinib or gefitinib; pertuzumab; ipilimumab; nivolumab; FOLFOX; capecitabine; FOLFIRI; bevacizumab; atezolizumab; selicrelumab; obinotuzumab or any combinations thereof. In one embodiment, prior treatment to said second line treatment, third-line, fourth-line or fifth-line treatment of cancer comprises ipilimumab and nivolumab. In one embodiment, prior treatment to said second line treatment, third-line, fourth-line or fifth-line treatment of cancer comprises FOLFOX, capecitabine, FOLFIRI/bevacizumab and atezolizumab/selicrelumab. In one embodiment, prior treatment to said second line treatment, third-line, fourth-line or fifth-line treatment of cancer comprises carboplatin/Nab-paclitaxel. In one embodiment, prior treatment to said second line treatment, third-line, fourth-line or fifth-line treatment of cancer comprises nivolumab and electrochemotherapy. In one embodiment, prior treatment to said second line treatment, third-line, fourth-line or fifth-line treatment of cancer comprises radiotherapy, cisplatin and carboplatin/paclitaxel.

In one embodiment, the treatment is first-line or second line treatment of HNSCC. In one embodiment, the treatment is first-line or second line treatment of recurrent/metastatic HNSCC. In one embodiment the treatment is first line treatment of recurrent/metastatic (1L R/M) HNSCC. In one embodiment, the treatment is first line treatment of 1L R/M HNSCC in a PD-L1 CPS (combined positive score) positive (CPS ≥1) patients. In one embodiment the treatment is second line treatment of recurrent/metastatic (2L R/M) HNSCC.

In one embodiment, the treatment is first-line, second-line, third-line, fourth-line or fifth-line treatment of PD-1/PD-L1-naïve HNSCC. In one embodiment, the treatment first-line, second-line, third-line, fourth-line or fifth-line treatment of PD-1/PD-L1 experienced HNSCC.

In some embodiments, the treatment results in one or more of increased tumor infiltrating lymphocytes including cytotoxic T cells, helper T cell and NK cells, increased T cells, increased granzyme B+ cells, reduced proliferating tumour cells and increased activated T cells as compared to levels prior to treatment (e.g. baseline level). Activated T cells may be observed by greater OX40 and human leukocyte antigen DR expression. In some embodiments, treatment results in upregulation of PD1 and/or PD-L1 as compared to levels prior to treatment (e.g. baseline level).

The present disclosure also relates to a method for treating or lessening the severity of a cancer selected from: brain (gliomas), glioblastomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer. In one embodiment, the cancer exhibits microsatellite instability (MSI). In one embodiment, the cancer inhibits high microsatellite instability (MSI-H).

By the term “treating” and grammatical variations thereof as used herein, is meant therapeutic therapy. In reference to a particular condition, treating means: (1) to ameliorate, or lessen the severity of, the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms or signs, effects or side effects associated with the condition or treatment thereof, (4) to slow the progression of the condition, that is to say prolong survival, or one or more of the biological manifestations of the condition and/or (5) to cure said condition or one or more of the biological manifestations of the condition by eliminating or reducing to undetectable levels one or more of the biological manifestations of the condition for a period of time considered to be a state of remission for that manifestation without additional treatment over the period of remission. One skilled in the art will understand the duration of time considered to be remission for a particular disease or condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the terms “cancer,” “neoplasm,” “malignancy” and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent Bcell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenström's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

In one embodiment, the methods of the present invention further comprise administering at least one neo-plastic agent or cancer adjuvant to said human. The methods of the present invention may also be employed with other therapeutic methods of cancer treatment.

Typically, any anti-neoplastic agent or cancer adjuvant that has activity versus a tumor, such as a susceptible tumor being treated may be co-administered in the treatment of cancer in the present invention. Examples of such agents can be found in Cancer Principles and Practice of Oncology by V. T. Devita, T. S. Lawrence, and S. A. Rosenberg (editors), 10^{th} edition (Dec. 5, 2014), Lippincott Williams & Wilkins Publishers.

It will be noted that embodiments of the method of treatment of cancer are also taken as embodiments of the agonist ICOS binding protein or antigen binding portion thereof for use in the treatment of cancer or use of an agonist ICOS binding protein or antigen binding portion thereof in the manufacture of a medicament for treating cancer and reciprocals thereof, in so far as it relates to dosages, treatment regimens and effects of said dosages and treatment regimens. It will also be noted that embodiments of the method of treatment of cancer, the agonist ICOS binding protein or antigen binding portion thereof for use in the treatment of cancer or use of an agonist ICOS binding protein or antigen binding portion thereof in the manufacture of a medicament for treating cancer are also taken as embodiments of the pharmaceutical composition, pharmaceutical formulation or pharmaceutical kit in so far as it relates to dosages, treatment regimens and effects of said dosages and treatment regimens.

The following examples are intended for illustration only, and are not intended to limit the scope of the invention in any way.

### EXAMPLES

### Example 1

H2L5 IgG4PE is a humanized IgG4 antibody selected for its potent binding, agonist activity against human ICOS and low/no depleting effects. The unique mechanistic profile of H2L5 IgG4PE offers an opportunity to investigate the antitumor potential of targeting a T cell co-stimulator alone and in combination with pembrolizumab. H2L5 IgG4PE comprises CDR sequences as shown in SEQ ID NOS: 1-6, and variable heavy chain and variable light chain sequences as shown in SEQ ID NO:7 and SEQ ID NO: 8, respectively.

Described herein is a first-time in human study evaluating the safety, pharmacokinetics (PK), pharmacodynamics (PD), and antitumor activity of H2L5 IgG4PE alone and in combination with pembrolizumab in selected solid tumors.

The study consists of dose escalation and cohort expansion phases; cohort expansion phases are ongoing in several tumor types.

The objectives of the study are as follows:

### Primary

Determine safety, tolerability, and maximum tolerated/administered dose of H2L5 IgG4PE as monotherapy and in combination with pembrolizumab.

### Secondary

Determine recommended H2L5 IgG4PE dose(s) for further exploration.
Evaluate preliminary antitumor activity; characterize PK; evaluate immunogenicity.

### Exploratory

Evaluate PD effects.
Explore associations between antitumor activity, PK and biomarkers in tissue and blood.

### Methods

The Study is a dose escalation (DE) and ongoing expansion phase study of H2L5 IgG4PE alone (Part 1) and in combination with pembrolizumab (Part 2). Modified toxicity probability interval informed DE decisions with >3 patients enrolled per dose level (DL). H2L5 IgG4PE is administered as intravenous infusion every 3 weeks (Q3W) f 200 mg pembrolizumab Q3W; treatment continues up to 2 years or until progression or unacceptable toxicity. Patients must have metastatic or relapsed invasive malignancy, measurable disease, received 5 lines of prior therapy in the advanced setting, adequate organ function, and no active autoimmune disease requiring treatment; PK/PD cohorts require pre-treatment and Day 43 on-treatment tumor biopsies. Primary objective is to determine safety, tolerability, and maximum tolerated (MTD) H2L5 IgG4PE dose.

### Patients—Key Inclusion Criteria

Histological or cytological documentation of advanced/metastatic or recurrent invasive malignancy.
Disease that has progressed after standard therapy for the specific tumor type, or for which standard therapy has proven to be ineffective, is intolerable, or is considered inappropriate, or if no further standard therapy exists
≤5 prior lines of therapy for advanced disease including both standard of care and investigational therapies.
Measurable disease according to RECIST v1.1 guidelines; Eastern Cooperative Oncology Group performance status 0-1; adequate organ function.
Agree to undergo a pre-treatment and on-treatment biopsy and have disease amenable to biopsy required in PK/PD dose expansion cohorts.

### Patients—Key Exclusion Criteria

Prior anticancer or investigational therapy within 30 days or five half-lives, whichever is shorter.
≥Grade 3 toxicity related to prior immunotherapy and led to treatment discontinuation.
History of invasive malignancy other than disease under study unless disease-free for 2 years.
Central nervous system (CNS) metastasis; exceptions include previously treated CNS metastasis that is asymptomatic and has no requirement for steroids at least 14 days prior to first dose of study treatment.
Active autoimmune disease that required systemic treatment within the last 2 years.
History of idiopathic pulmonary fibrosis, pneumonitis requiring steroids, interstitial lung disease, or organizing pneumonia.

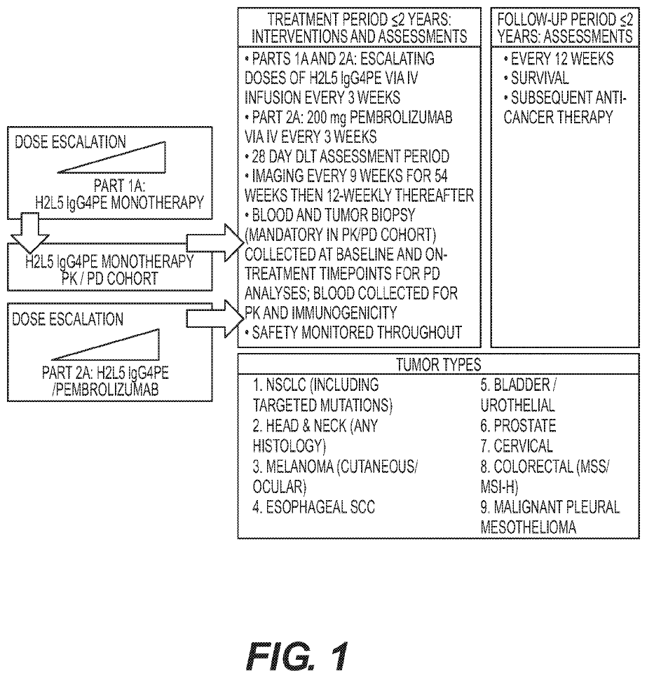

### The Study Design is Shown in FIG. 1

Accelerated titration design for the first 3 dose levels in Part 1A; 1 patient enrolled at each dose level.
Modified toxicity probability interval method informed subsequent dose escalation decisions in Parts 1A/2A (minimum 3 patients per dose level).
Starting dose of 0.001 mg/kg: the projected human dose based on the minimally anticipated biologic effect observed in preclinical studies.

This is a first time in human (FTIH), open-label, multicenter study designed to investigate the safety, tolerability, pharmacology, PK, preliminary clinical activity, and establish a recommended dose of H2L5 IgG4PE for further exploration.

As illustrated in FIG. 1, the study is conducted in two parts (Part 1 H2L5 IgG4PE monotherapy and Part 2 H2L5 IgG4PE combination therapy) whereby each part consists of a dose escalation phase followed by a cohort expansion phase.

Part 1A dose escalation phase evaluates escalating weight-based dose levels of monotherapy H2L5 IgG4PE administered intravenously once every three weeks (Q3W) to subjects with selected relapsed and/or refractory solid tumors. Based on safety and tolerability, and the PK/pharmacodynamic characteristics of the molecule, recommended monotherapy dose level or dose levels may be further investigated in expansion cohorts (Part 1B).

Part 2A pembrolizumab combination dose escalation phase is initiated when a monotherapy dose level of H2L5 IgG4PE has been deemed safe and has demonstrated consistent, dose-responsive pharmacodynamic activity; two dose levels below this dose level will become the starting dose investigated in combination with a 200 mg fixed dose of pembrolizumab.

These combinations evaluated in Part 2A is investigated in subjects with selected, relapsed and/or refractory solid tumors.

While Part 1B and Part 2B expansion cohorts may initiate with H2L5 IgG4PE weight-based dosing, a transition to fixed dosing may be made.

Seamless design is implemented to combine dose escalation with dose expansion, based on toxicity and efficacy (Pan H, Fang X, Liu P, et al. A phase I/II seamless dose escalation/expansion with adaptive randomization scheme (SEARS). Clinical Trials. 2013; 0:1-11). In both Part 1 and Part 2, the dose expansion phase may start before the dose escalation phase is completed. All available safety and tolerability data from subjects in dose expansion is incorporated into dose escalation decision making. The basis of the decision to initiate expansion of a dose level/dose will consider following graduation rules:
Established safety and tolerability;
Preliminary PK/pharmacodynamic characteristics (i.e., measures of target engagement and functional effects such as receptor occupancy and cytokine release) and/or
Preliminary antitumor activity.

Once a dose level(s) passes the graduation rules the selected dose(s) may enter into the expansion phase for further investigation following approval of the Steering Committee; alternate H2L5 IgG4PE schedules or drug sequencing may be investigated in the expansion phase. In addition, dose levels under investigation in the ongoing monotherapy dose escalation phase may incorporate information, such as safety data, from subjects who were accrued to the expansion phase. Randomization and/or futility rules may be incorporated if appropriate in expansion phase to optimize the dose allocation based on evaluations of safety and antitumor activity. The details of randomization schema for expansion cohorts will be documented before the initiation of expansion cohort; details of the futility rules will be documented in the RAP before initiation of interim analyses (Pan H, Fang X, Liu P, et al. A phase I/II seamless dose escalation/expansion with adaptive randomization scheme (SEARS). Clinical Trials. 2013; 0:1-11).

The overall study will enroll approximately 500 subjects diagnosed with solid tumor malignancies.
In the dose escalation phases of the study, and in the pharmacokinetic (PK)/pharmacodynamic (PD) expansion cohorts the solid tumor types selected for inclusion include bladder/urothelial cancer, cervical cancer, colorectal cancer (CRC), esophageal cancer with squamous cell histology, head and neck (HN) cancer, melanoma, malignant pleural mesothelioma (MPM), non-small-cell lung cancer (NSCLC), and prostate cancer.
In the cohort expansion phases of the study (Part 1B and Part 2B), several expansion cohorts have been defined by tumor histology or by a specific characteristic such as tumors exhibiting high microsatellite instability (MSI-H), deficiency in DNA mismatch repair (dMMR) processes, or viral-mediated pathology; enrollment in these cohorts is not limited to the tumor types/histologies in the aforementioned list (defined as tumor agnostic).
Additional expansion cohorts may enroll subjects with a specific tumor type selected from the aforementioned list or from a tumor type/histology not protocol-defined; the basis for the selection will be evidence-based and by an amendment to the protocol to define the cohorts.

The overall study size may extend beyond 500 by a protocol amendment if data from expansion phases support extended enrollment or additional combinations are investigated.

Assessment of disease status is performed by the Investigator in accordance with Response Evaluation Criteria In Solid Tumors (RECIST) v1.1 and Immune Related (ir) RECIST. A decision to discontinue treatment due to disease progression is based upon irRECIST; primary efficacy endpoint analysis will use irRECIST. Scans are collected centrally and stored to allow for the option of central review.

TABLE 2
Study Treatment

| | | |
|---|---|---|
| Product Name: | H2L5 IgG4PE | Pembrolizumab |
| Product | Humanized anti-ICOS | Humanizedanti-PD-1 |
| Description | IgG4 mAb | IgG4 mAb |
| Dosage form/ | 10 mg/mL solution | 100 mg/4 mL |
| strength: | | solution/25 mg/mL |
| Planned dosage | 0.001 to 3 mg/kg | 200 mg |
| level(s): | | |
| Route of | IV infusion | IV infusion |
| Administration | | |
| Dosing | Administer diluted | Administer diluted |
| instructions/ | product/once Q3W | product/once Q3W |
| Frequency: | | |
| Part of Study | 1 and 2 | 2 |
| Manufacturer | GSK | Merck |

### H2L5 IgG4PE Fixed Dose Rationale

In Part 1A (monotherapy) and Part 2A (combination with pembrolizumab) H2L5 IgG4PE was administered on body weight-based dosing. Fixed doses may be tested in the expansion cohorts and in the safety run-in phase with chemotherapy combinations, assuming a typical median weight of 80 kg.

Therapeutic monoclonal antibodies are often dosed based on body-size due to the concept that this reduces inter-subject variability in drug exposure. However, body-weight dependency of PK parameters does not always explain the observed variability in the exposure of monoclonal antibodies (Zhao X, Suryawanshi, S; Hruska, M. Assessment of nivolumab benefit-risk profile of a 240-mg flat dose relative to a 3 mg/kg dosing regimen in patient with advanced tumors. Annals of Oncology. 2017; 28:2002-2008). The advantage of body-weight based versus fixed dosing in this study was evaluated through population PK modelling and simulation efforts. A preliminary population PK model was developed from monotherapy dose escalation (data up to doses of 1 mg/kg; n=19 subjects).

Simulations were performed by considering body weight distribution in the simulations were based on the observed distribution in the preliminary dataset. At the 5th percentile of body weight (40-47 kg), there was a 70-100% increase in median steady-state AUC(0-); H2L5 IgG4PE exposures higher than these increases have been evaluated in the current Phase 1 study with the 3 mg/kg dose regimen. At the 95th percentile of body weight (107-118 kg), there was a 23-32% decrease in median steady-state AUC (0-) as compared to the median 80 kg exposure providing adequate receptor occupancy (RO) with the minimal lowering of exposure. A similar outcome is expected for steady-state Cmax and trough concentrations between body weight-based and fixed dosing.

Overall, these preliminary population PK simulations indicate that using fixed dosing would result in a similar range of exposures as that of body weight-based dosing. Also, fixed dosing offers the advantage of reduced dosing errors, reduced drug wastage, shorten preparation time, and improve ease of administration. Thus, switching to a fixed dose based on a reference body weight of 80 kg is reasonable and appropriate.

The fixed dose equivalents of the weight-based H2L5 IgG4PE dose levels using 80 kg weight are presented in Table 3.

TABLE 3
H215 IgG4PE Fixed Dose Calculations

| Dose | H2L5 IgG4PE | H2L5 IgG4PE |
|---|---|---|
| Level | mg/kg | mg |
| 1 | 0.001 | 0.08 |
| 2 | 0.003 | 0.24 |
| 3 | 0.01 | 0.8 |
| 4 | 0.03 | 2.4 |
| 5 | 0.1 | 8.0 |
| 6 | 0.3 | 24.0 |
| 7 | 1.0 | 80.0 |
| 8 | 3.0 | 240.0 |

### Results

In the DE phase and the PK/PD cohort, 98 patients enrolled: Part 1: 22 in DE and 40 in the PK/PD cohort; Part 2: 36 in DE. Majority of patients had microsatellite stable colorectal carcinoma (26%) and 2 baseline target lesions (57%); 37% received 3 prior lines of therapy in the advanced setting and 31% prior anti-PD-1/L1 therapy. In Part 1 (n=62), 22 patients (35%) had at least one treatment-related adverse event (TR-AE). The most frequent TR-AEs (3 patients) were fatigue (15%), aspartate aminotransferase (AST) elevations (5%) and diarrhea (3%); AST elevations were the most frequent Grade 3/4 TR-AE (N=2 [3%]). In Part 2, 15 patients (42%) had at least one TR-AE; most frequent TR-AEs were AST elevations (8%) and pyrexia (8%); no Grade 3/4 TR-AEs occurred in >1 patient. One dose limiting toxicity (DLT) occurred in DE: Grade 3 pneumonitis in a Part 2 patient treated at the top H2L5 IgG4PE DL of 3 mg/kg which led to discontinuation of both drugs. In the PK/PD cohort, liver enzyme increases in 1 patient (H2L5 IgG4PE 3 mg/kg) were DLTs and the only TR AEs leading to treatment discontinuation. Disease progression was the primary reason for treatment discontinuation (92%). Approximate dose proportional increases in systemic H2L5 IgG4PE concentrations over 0.01-3 mg/kg DLs were observed. At DLs ≥0.3 mg/kg, ICOS receptor occupancy was 75% across the dosing interval. On-target PD effects in tumor infiltrating lymphocytes and clinical activity were observed in Part 1 and 2; including in anti-PD-1/L1 experienced patients.

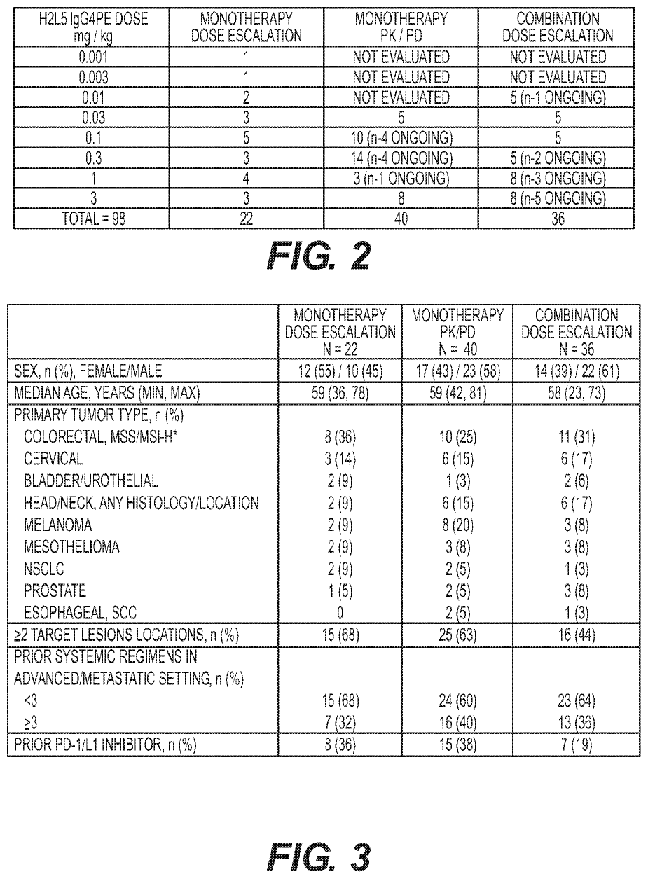

FIG. 2 shows patient disposition by cohort and dose. FIG. 3 shows patient and disease characteristics.

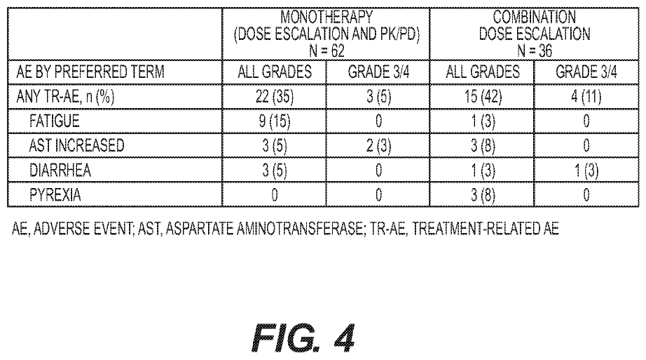

FIG. 4 shows treatment-related AEs (in >3 patients).

Treatment-Related Safety
Treatment-related dose-limiting toxicities leading to discontinuation reported at 3 mg/kg H2L5 IgG4PE dose level:
1 patient in monotherapy dose escalation cohort experienced Grade 3/4 elevations in alanine aminotransferase, aspartate aminotransferase, alkaline phosphatase, bilirubin, gamma-glutamyl transpeptidase, impaired liver function (serious) and Grade 1 amylase and G3 lipase.
1 patient in combination cohort experienced Grade 3 pneumonitis.
Serious adverse events (SAEs) in monotherapy group: 1 patient (3 mg/kg) had impaired liver function (Grade 3).
Confounded by progression in liver metastases and biliary tract obstruction that required stenting.
SAEs in H2L5 IgG4PE/pembrolizumab group: 4 patients had SAEs.
Grade 5 pulmonary hypertension (H2L5 IgG4PE 0.1 mg/kg/pembrolizumab 200 mg): occurred after patient off study treatment for 2 months; patient had co-morbidities including hepatitis, pancreatitis and methemoglobinemia (dapsone related).
Grade 3 diarrhea (H2L5 IgG4PE 0.3 mg/kg/pembrolizumab 200 mg): resolved with 5 steroids.
Grade 3 hypotension (H2L5 IgG4PE 1 mg/kg/pembrolizumab 200 mg): occurred after study treatment infusion; resolved and did not result in treatment discontinuation.
Grade 3 pneumonitis and Grade 3 lower respiratory infection, which were not concurrent (H2L5 IgG4PE 3 mg/kg/pembrolizumab 200 mg): pneumonitis resolved with steroids; lower respiratory infection may be a flare due to pneumonitis; resolved with antibiotics and steroids.

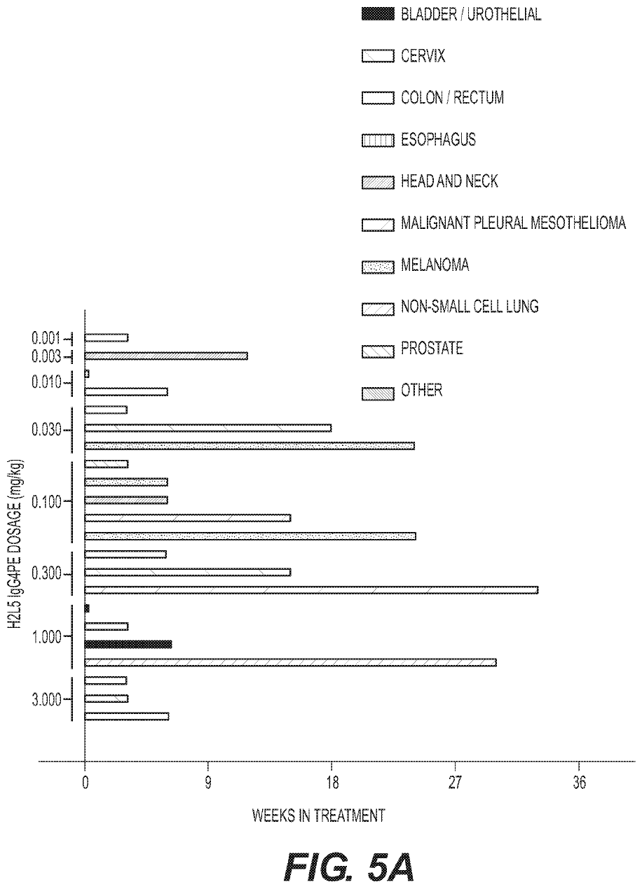

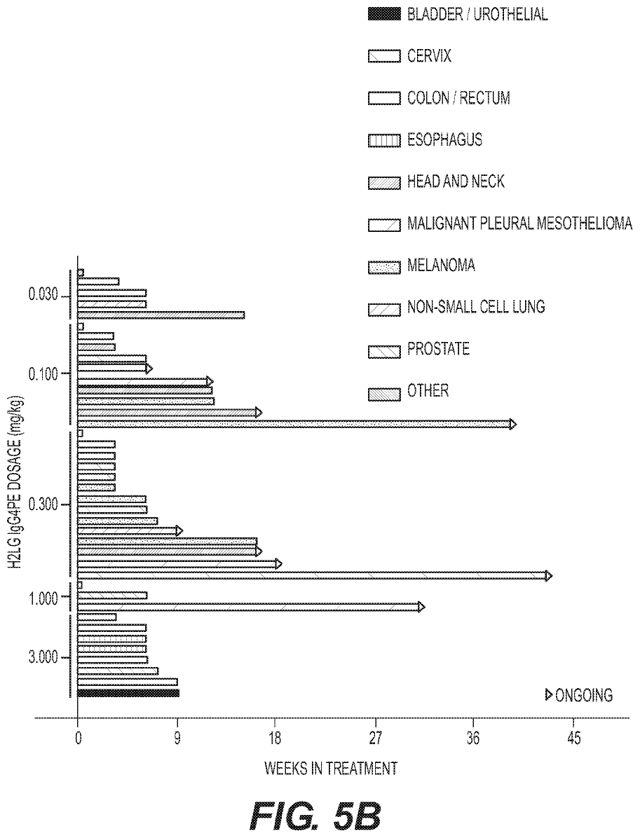

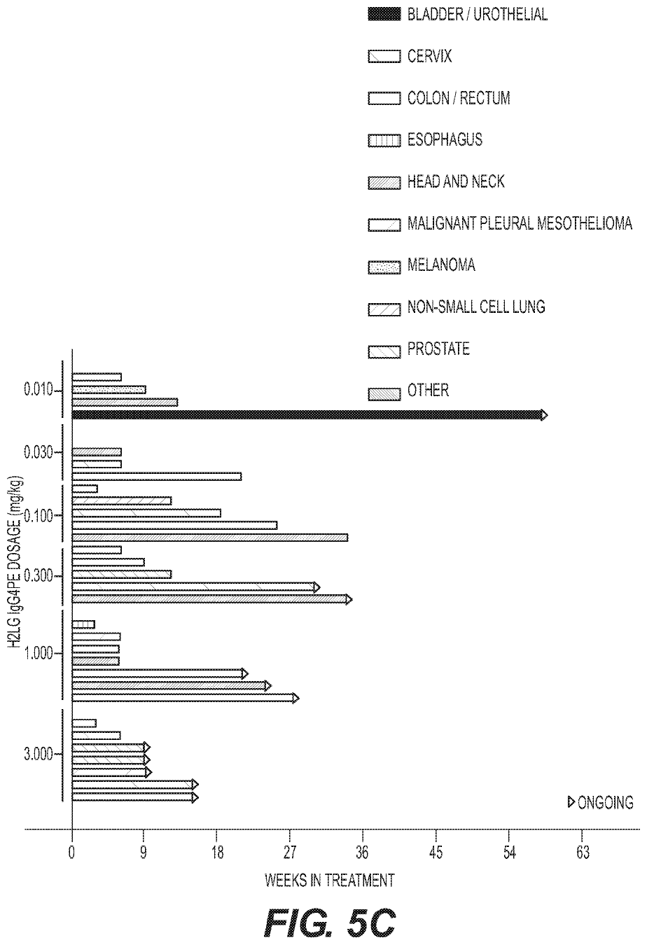

FIGS. 5A-5C show duration of study treatment: individual patient data.

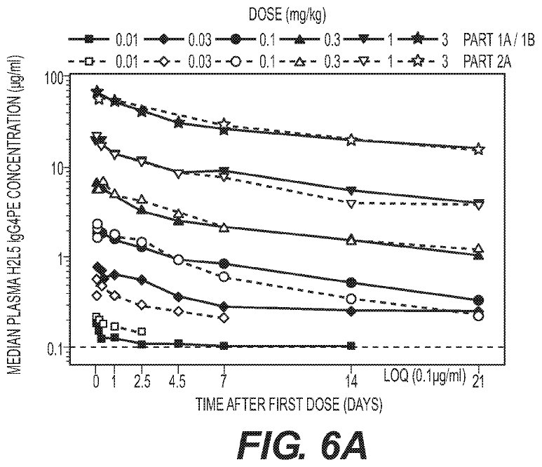

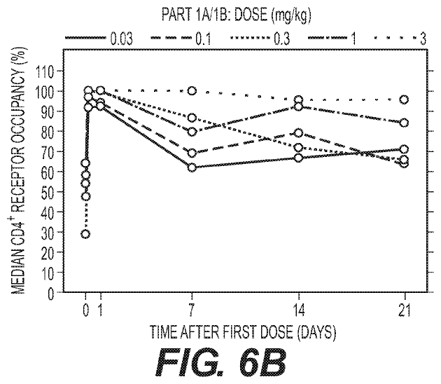

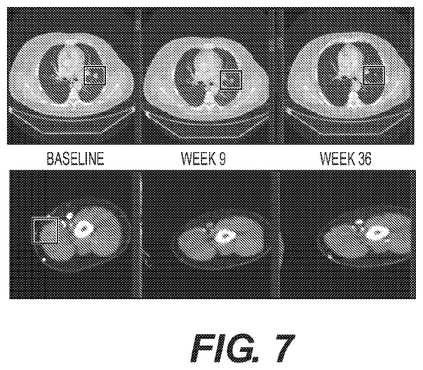

FIGS. 6A-6B show PK and receptor occupancy.

FIGS. 7-10 show results from patient case studies.

### Patient 1: H2L5 IgG4PE Monotherapy (FIG. 7)

### History:

53Y Male; Stage IIIc nodular melanoma [BRAF/cKIT mutation negative].
Prior regimens: ipilimumab/nivolumab ˜2 months; nivolumab ˜1 year, SD (stable disease) best response.
Disease Burden: 5 target lesions (LN, Lung, SubQ): SoD (sum of diameters)=225 mm. Multiple non target lesions.

### Study Treatment:

H2L5 IgG4PE monotherapy to Week 48; 0.1 mg/kg×3 doses Q3W, then 1 mg/kg Q3W.
H2L5 IgG4PE Q3W+200 mg Q3W pembrolizumab combination ongoing.
   FIG. 7 shows CT images from baseline (prior to initiation of H2L5 IgG4PE monotherapy) and at on-study treatment assessment intervals of a lung lesion and subcutaneous lesion showing tumor response to treatment. There was tumor regression in lung lesion an a complete response/regression in the subcutaneous lesion that was durable.

Tumor biopsies collected after 43 days on-treatment when compared with tumor tissue obtained at screening showed
greater number of T cells,
increase in granzyme-B expressing CD8 Tc cells,
increase in PD1 expressing T cells and
greater number of proliferating T cells while
fewer proliferating tumor cells.

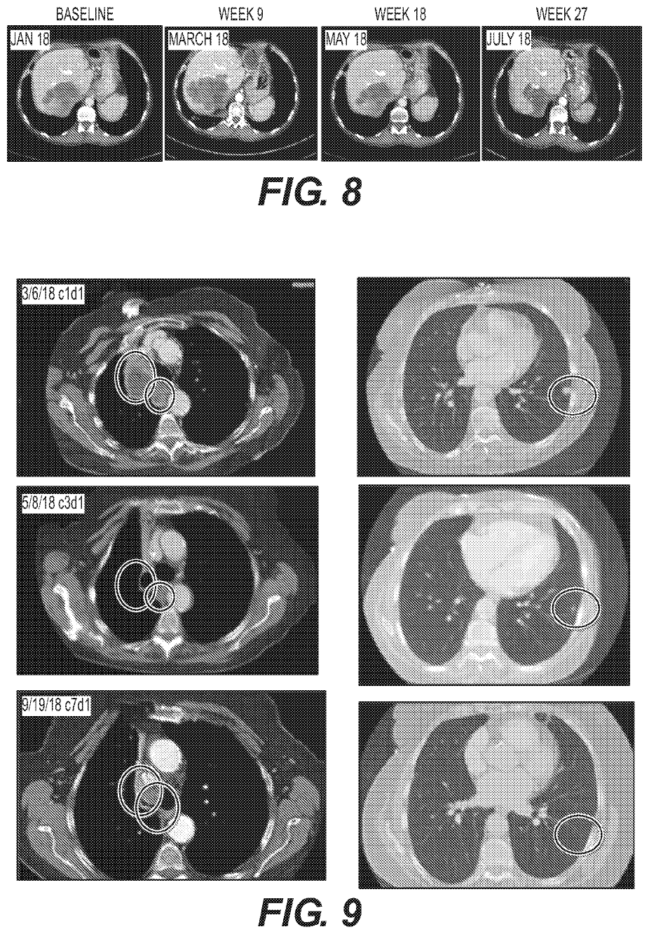

### Patient 2: H2L5 IgG4PE Plus Pembrolizumab Combination Therapy (FIG. 8)

### History:

53Y Female, Diagnosis: Stage IV KRAS mutant, MSI-H CRC.
Prior regimens: FOLFOX; capecitabine; FOLFIRI/bevacizumab; atezolizumab/selicrelumab ˜3 months, BoR (best overall response): PD (progressive disease); Anti-CEA ADC ˜2 months, BoR: PD; R0695688/obinutuzumab ˜2 months, BoR: PD.

### Study Treatment:

H2L5 IgG4PE 1 mg/kg Q3W+ pembrolizumab 200 mg Q3W ongoing.
   FIG. 8 shows CT images from baseline and at on-study treatment assessment intervals showing a large liver lesion that at Week 9 increased in size then at subsequent assessments decreased in response to study treatment, representing a case of pseudoprogression.
   Patient 3: PD Changes—Tumor Infiltrating Lymphocytes Squamous Cell Carcinoma of the Head and Neck—H2L5 IgG4PE 0.3 mg/kg Q3W+Pembrolizumab 200 mg Q3W

Post treatment sample showed an increase in Granzyme B+ and PD-L1+ cells compared with pre-treatment samples. Tumor tissue at screening or pre-treatment was compared with fresh tumor biopsies obtained at week 6 on-treatment. Changes in tumor immune infiltrates or TIL was evaluated by multiplexed immunofluorescence platform called MultiOmyx using a panel of 16 markers. The TIL analysis of week 6 tumors compared to the pre-treatment tumor tissue for this patient demonstrates increase in Granzyme B+ T cells and PD-L1+ cells.

### Conclusions

H2L5 IgG4PE alone and in combination with pembrolizumab was well tolerated in patients with advanced solid tumours at the 0.001-3 mg/kg dose range.
Maximum tolerated dose was not reached; maximum administered dose was 3 mg/kg H2L5 IgG4PE.
Majority of AEs (adverse events) were Grade 1/2 and not attributed to study treatment.
AEs leading to discontinuation occurred in 1 patient each in monotherapy (n=62 patients) and combination (n=36 patients) at the highest dose level.
Dose proportional increases in H2L5 IgG4PE concentrations.
PK/PD analysis showed ≥75% total ICOS receptor saturation across dosing interval at H2L5 IgG4PE dose levels ≥0.3 mg/kg.
A range of doses (0.1-1 mg/kg) have shown biological and clinical activity (including in patients with prior anti-PD-1/L1 exposure). These doses are being investigated further in expansion cohorts to establish the recommended H2L5 IgG4PE dose.
Preliminary biological and clinical data support the mechanism of action of a non-depleting ICOS agonist as a clinical target.
Doses over 0.1 mg/kg are being investigated further in expansion cohorts to establish the recommended H2L5 IgG4PE dose.

### Squamous Non-Small-Cell Lung Cancer (NSCLC) Patient

FIG. 9 shows scans of a squamous NSCLC patient, which show a response to H2L5 IgG4PE (0.3 mg/kg Q3W)/pembrolizumab (200 mg Q3W) combination therapy.
Stage IV Squamous Cell Carcinoma to lungs, LN (Lymph Node) PD-L1=0%, TMB (tumor mutation burden)=8 mt/MB (mutation/megabase), ICOS-high (GSK LDT lab developed test)
   Note: PD-L1 with Dako22C3; TMB with FoundationOne
Foundation One gene panel: PIK3CA, TSC1, SOX2, BCL2L2, CARD11, MCL1, PRKCI, SPTA1, TERC, TP53
Prior treatment with Carboplatin/Nab-Paclitaxel 9/17-2/18
Partial response (PR) on H2L5 IgG4PE+Pembro (63%)
2/21/19 cycle 17 day 1

### Example 2

Example 2 describes pharmacokinetics/pharmacodynamics (PK/PD) exposure-response characterization of H2L5 IgG4PE from the study described in Example 1. H2L5 IgG4PE is an agonist IgG4PE antibody against inducible co-stimulatory receptor (ICOS) with immune stimulating and anti-neoplastic activity. The study described Example 1 is the first in human study investigating H2L5 IgG4PE alone and in combination, including first-line recurrent/metastatic (1L R/M) HNSCC in combination with pembrolizumab.

### Methods

Safety, PK, PD, and preliminary antitumor activity of H2L5 IgG4PE were evaluated at doses from 0.001 to 10 mg/kg every 3 weeks (Q3W). Blood samples collected prior to dosing and select time points on-study were evaluated for PK and PD effects on lymphocytes and ICOS receptor occupancy (RO). Tumor biopsies at Screening and Week 6 were evaluated for changes in tumor immune infiltrates (TIL) by a multiplexed immuno-fluorescence and gene expression platforms.

### PK Analysis

A preliminary population PK data set was constructed with all pooled concentration-time data
Serial plasma samples were collected throughout; PK samples were assayed by validated ELISA assay and concentration-time data was modeled using nonlinear mixed effects, as implemented in NONMEM™.

### Pharmacodynamic (PD) Analysis

Flow cytometry was performed instream throughout the study to evaluate ICOS receptor occupancy (RO) with H2L5 IgG4PE.
For PK/PD and expansion cohorts, tumor tissue was collected at predose and at Week 6 for evaluation of overall TIL, changes in activation, proliferation and gene expression changes.
Exposure measure for PK/PD analyses defined as Week 6 pre-dose trough concentration derived from population PK model.
Evaluation of gene expression changes in the TME were performed using the Nanostring nCounter™ platform.
Multiomyx™ multiplexed immunofluorescence was used to characterize the immune phenotype of the TIL.

### HNSCC Exposure-Response Analysis

Exposure-efficacy analysis was carried out on participants with HNSCC naïve to prior anti-PD 1/L1 therapy who had received the study drug in either the Part 2A dose escalation or Part 2B HNSCC cohort.
Exploratory regression analysis was performed to evaluate potential associations between H2L5 IgG4PE exposure and change in tumor sum of longest diameters (SLD). Survival analyses were premature.
Exposure measure for exposure-response analyses defined as first dose area under the curve, derived from population PK model.
Investigator assessed overall response rate (ORR) and disease control rate (DCR) per IRECIST, was summarized by binned exposure estimates and described using a conventional logistic regression model.

### Results

Preliminary PK disposition of H2L5 IgG4PE showed low clearance, limited central volume of distribution, and mean systemic half-life of 19 days, which is consistent with that of other humanized mAbs. Evidence of target engagement and tumor size reduction were observed in the 1L R/M HNSCC expansion cohort at 0.3 mg/kg with concomitant 200 mg pembrolizumab. Dose and concentration-RO analyses suggest 0.1 mg/kg H2L5 IgG4PE maintains high RO (≥70%) on peripheral CD4+ and CD8+ T cells. Quantitative TIL evaluation of paired tumor biopsies demonstrates potentially favorable immune microenvironment in the tumor at exposures observed in subjects treated with 0.3 mg/kg dose. TIL and gene expression data from tumor RNA demonstrate non-linear, dose-dependent changes in select markers of immune activation. Clinical exposure-response assessments reveal no difference in baseline-to-Week 9 target lesion change across exposures in the 1L R/M HNSCC expansion cohort. Likewise, cross-cohort pooled exposure-response analysis of AEs of Grade ≥2 severity demonstrates similar safety outcomes across the exposures/doses. Population PK modeling suggests fixed doses maintain exposures within established safety bounds.

### Pharmacokinetics and Target Engagement

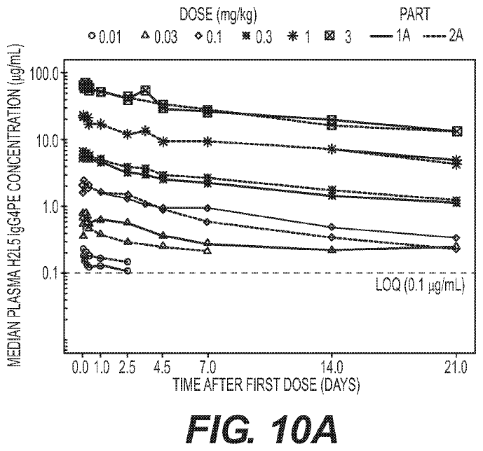

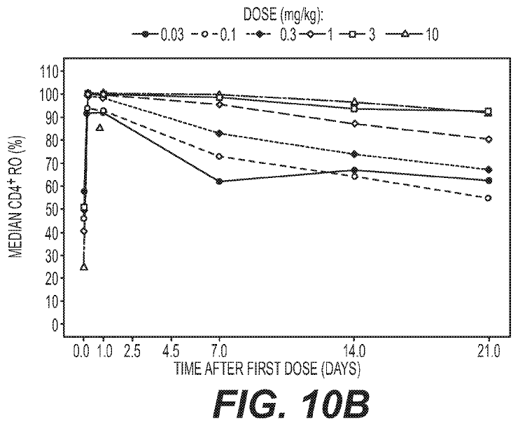

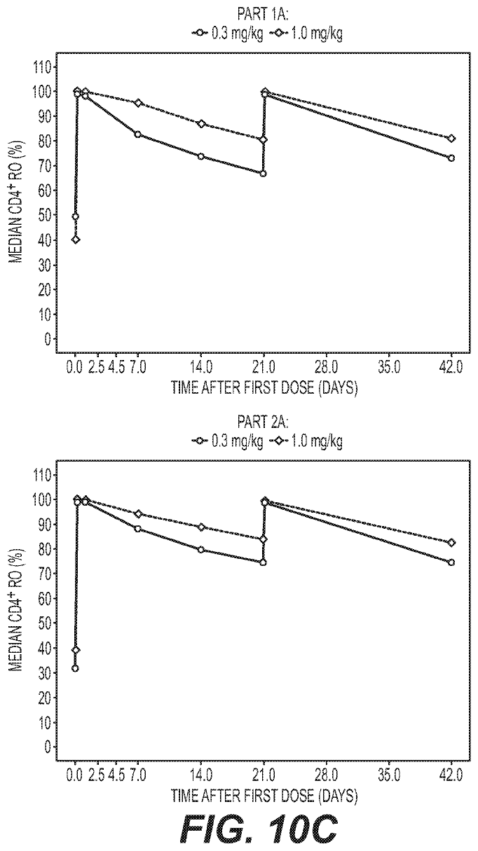

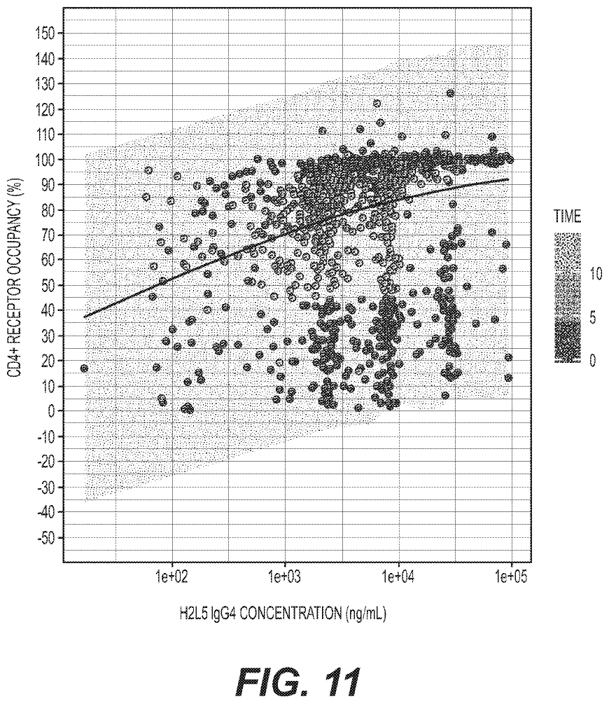

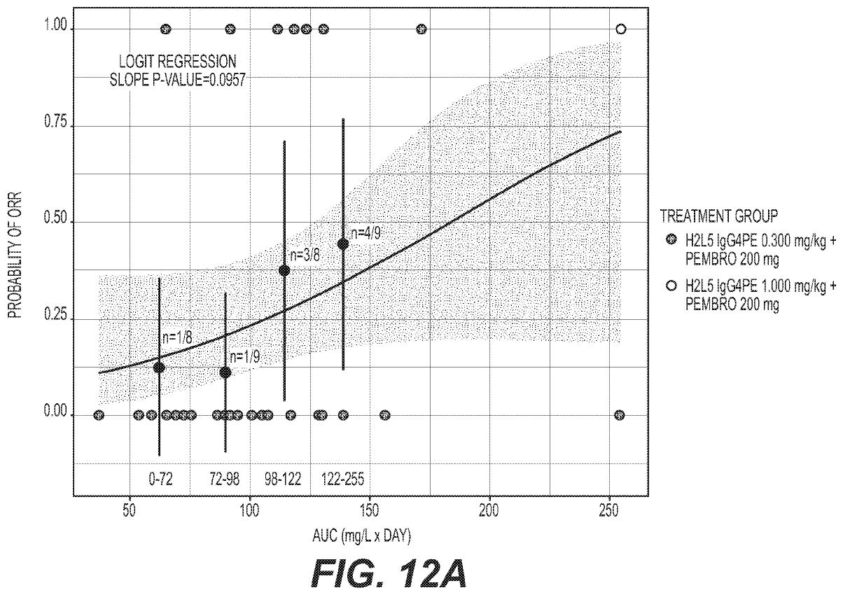

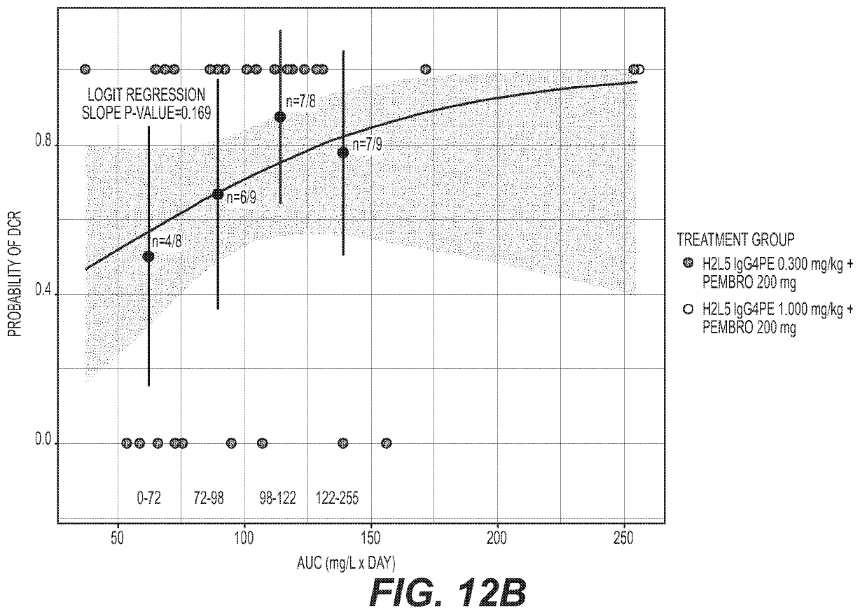

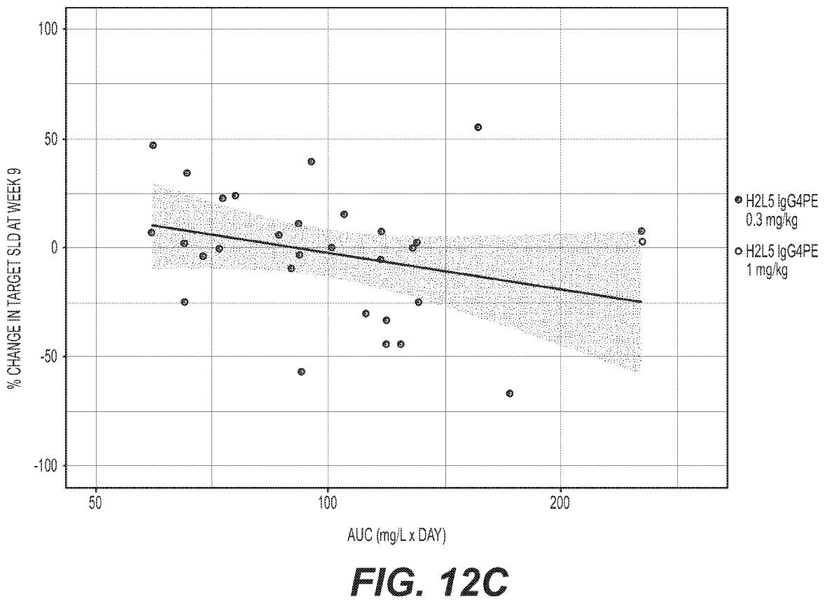

PK and target engagement characteristics of H2L5 IgG4PE are similar to prior reports, with a population clearance estimate of ˜0.27 L/day and central volume estimate of ˜3.6 L, and limited impact of bodyweight on systemic exposure.
Plasma concentrations of H2L5 IgG4PE increase in a dose-proportional manner with no apparent pembrolizumab interaction (FIG. 10A), while ICOS RO was maintained above ˜70% with H2L5 IgG4PE doses of 0.1 mg/kg and higher (FIG. 10B).
Minimal differences in RO are observed for CD4⁺ with H2L5 IgG4PE doses of 0.3 mg/kg and 1.0 mg/kg (FIG. 10C), with similar results for CD8⁺ (data not shown). However, there was large variability in RO for doses <1.0 mg/kg (FIG. 11).

### Exposure-Response Characterization

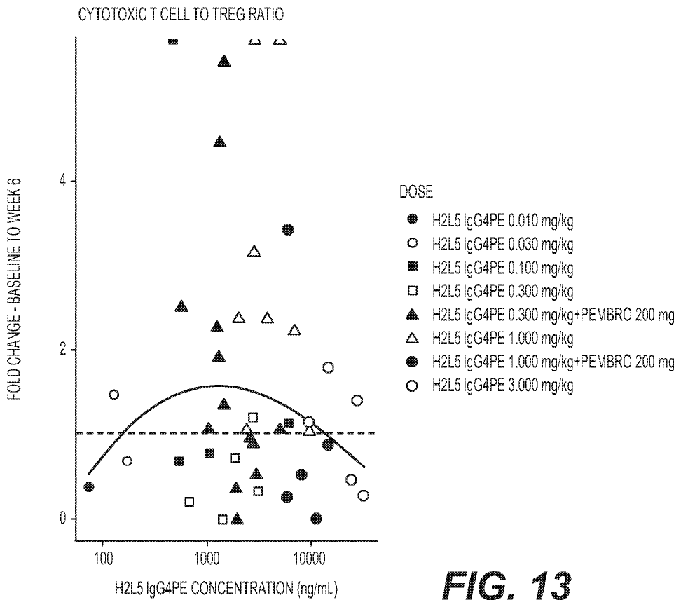

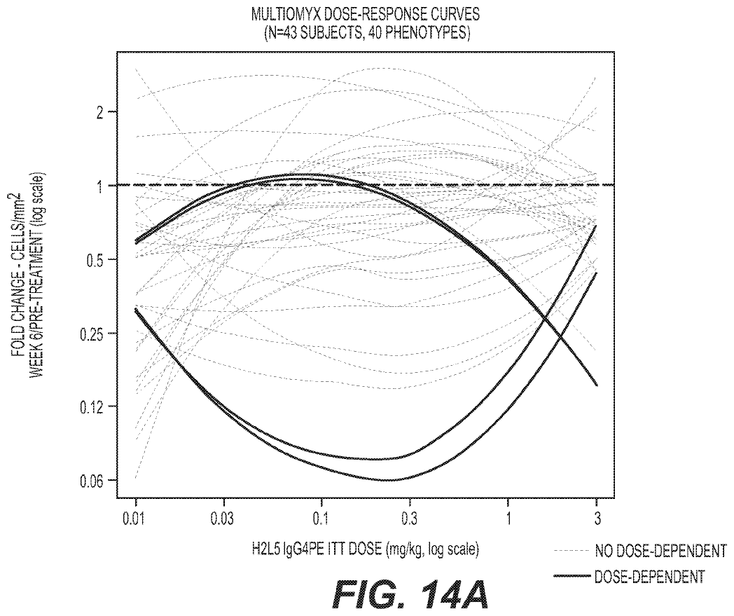

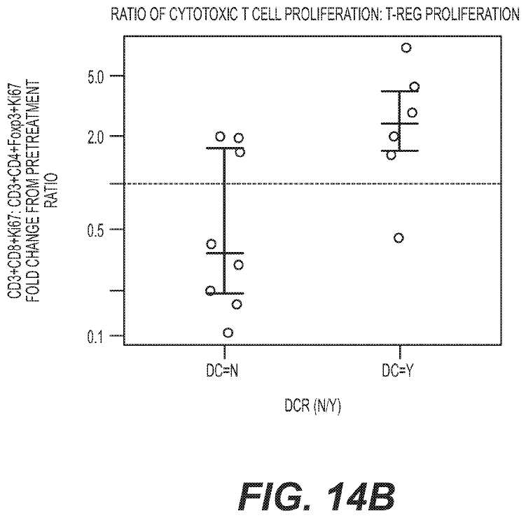

Evidence of target engagement and tumor size reduction is observed in the recurrent/relapsed head and neck squamous cell carcinoma (R/R HNSCC) expansion cohort with H2L5 IgG4PE 0.3 mg/kg and 200 mg pembrolizumab.
A potential association between exposure (AUC) and unconfirmed Best Overall Response (ORR), DCR (disease control rate), and percent change in SLD at week 9 is observed in HNSCC. Each open/shaded circle represents a patient in the head and neck expansion cohort. None of the slope estimates from these three regression analyses are statistically significant (p-value >0.05; FIG. 12).
   MultiOmyx—Multiplexed Immunofluorescence Based Evaluation of TIL and Gene Expression Data to Demonstrate Pharmacodynamic Changes in the Tumor with H2L5IgG4PE+/−Pembrolizumab.
Quantitative evaluation of TILs in paired tumor biopsies demonstrates on-study changes in TILs follow a non-linear, exposure/dose-dependent pattern.
Changes in select immune activation markers favors a greater cytotoxic T cell to regulatory T cell ratio with H2L5 IgG4PE exposures of 1000-10000 ng/ml at C_{trough} which corresponds to doses between ˜0.3 mg/kg to 1 mg/kg (FIG. 13).
Non-monotone dose-dependent changes in total TIL as well as other activation and proliferating T cell phenotypes were detected in on-treatment biopsies when compared to baseline in MultiOmyx™ immunofluorescence data with H2L5 IgG4PE 0.3 mg/kg and higher doses (FIG. 14A)
Gene expression changes in the tumor show a non-linear dose response trend with the highest increases at ≥0.1 mg/kg and greatest reductions at <1 mg/kg (data not shown)
The ratio of cytotoxic T cell proliferation (CD3⁺CD8⁺Ki67⁺) over regulatory T cells proliferation (CD3⁺CD4⁺FOXP3⁺Ki67⁺) was higher in Week 6 on-treatment biopsies when compared to pre-treatment tumor samples for subjects at 0.3-1 mg/kg doses of H2L5 IgG4PE who experienced disease control (DC) benefit when compared to subjects that did not experience disease control (FIG. 14B). In a similar pattern, the ratio of cytotoxic T cells (CD3+CD8+) over regulatory T cells (CD3+CD4+Foxp3+) was also higher in week 6 on-treatment biopsies when compared to pre-treatment tumor samples for subjects at 0.3-1 mg/kg doses of H2L5 IgG4PE who experienced disease control (DC) benefit when compared to subjects that did not experience disease control (data not shown).

### Patient Case Study

### Patient 4: H2L5 IgG4PE Monotherapy

### History

### Diagnosis:

Initial diagnosis (Dx): March 2013, BRAF negative, N/KRAS mutation positive Stage Ib superficial spreading melanoma.
Dx (diagnosis) with metastasis: January 2013

### Prior Regimens:

Nivolumab (advanced/metastatic, August 2017-June 2018)
Electrochemotherapy (March 2018)

### Study Treatment:

Cycle 1 day 1 (C1D1)—24 Jul. 2018; H2L5 IgG4PE monotherapy at 1 mg/kg Q3W.

The post treatment sample showed:
Higher TIL including cytotoxic, helper T cells and NK cells
More Granzyme B+ T cells and less proliferating tumour cells
Increase in activated T cells as observed with greater OX40 and HLADR expression
Upregulation of PD1 and PD-L1 upon H2L5 IgG4PE treatment

### Patient Case Study

### Patient 5: H2L5 IgG4PE Monotherapy

### History

### Diagnosis

49Y female, Stage III parotid gland cancer with mucoepidermoid histology (diagnosed Oct. 20, 2014) and metastasis (diagnosed July 2016).
Prior regimens: radiation therapy (December 2014-January 2015); doxorubicin/cyclophosphamide (February 2015-April 2015); paclitaxel (adjuvant) (May 2015-May 2016).

### Study Treatment:

H2L5 IgG4PE monotherapy 0.3 mg/kg Q3W; discontinued after Week 24 due to disease progression.
Crossover Week 27 to H2L5 IgG4PE 0.3 mg/kg Q3W in combination with pembrolizumab 200 mg Q3W; discontinued at Week 36 due to PD (disease progression)).
Immune phenotyping of the tumors using multiplexed immunofluorescence exhibits increase in functional markers representing TIL activation, cytotoxic function and proliferation in on-treatment tumor biopsy for subject number compared to their pretreatment samples.

### Post Treatment Sample Showed:

More TIL including cytotoxic, helper T cells and NK cells
More Granzyme B+ T cells and less proliferating tumor cells
More activated T cells as observed with greater OX40 and HLADR expression
Upregulation of PD1 and PD-L1 upon H2L5 IgG4PE treatment

### Conclusions

H2L5 IgG4PE PK disposition is consistent with other humanized monoclonal antibodies, with low clearance and limited central volume of distribution. H2L5 IgG4PE PK was not affected by pembrolizumab.
Evidence of target engagement and tumor size reductions is demonstrated in patients with R/R HNSCC and Melanoma treated with 0.3-1.0 mg/kg doses of H2L5 IgG4PE.
A H2L5 IgG4PE dose range of 0.3-1.0 mg/kg is associated with a favorable tumor microenvironment as demonstrated by increases in CD8:Treg ratio and their proliferation changes. The data provide further pharmacological evidence of agonist stimulation of the ICOS receptor at these doses which could translate to clinical benefit.
Overall, the current PK and non-monotone PD data provides evidence of H2L5 IgG4PE target engagement and biological activity at clinically tolerable doses and support continued exploration of H2L5 IgG4PE including ranges 0.3-1.0 mg/kg, or comparable fixed dose range, in future studies.

### Example 3

Example 3 describes the preliminary efficacy and safety findings from the study described in Example 1 of H2L5 IgG4PE, used alone and in combination with pembrolizumab in HNSCC (head and neck squamous cell carcinoma), in PD-1/L1-experienced and PD1/L1-naïve patients respectively.

### Study Objectives

Study objectives and eligibility criteria have been described in Example 1.
The objectives of the analyses presented here are:
   Primary: Determine the safety, tolerability of H2L5 IgG4PE as monotherapy and in combination with pembrolizumab in patients with HNSCC.
   Secondary: Evaluate the anti-tumour activity of H2L5 IgG4PE as monotherapy and in combination with pembrolizumab by iRECIST.
Exploratory: The PD effects of H2L5 IgG4PE monotherapy in the blood and tumor which include, but are not limited to, receptor occupancy, immune phenotyping, changes in TIL and gene expression have been evaluated in separate analyses.

### Methods

### Study Design

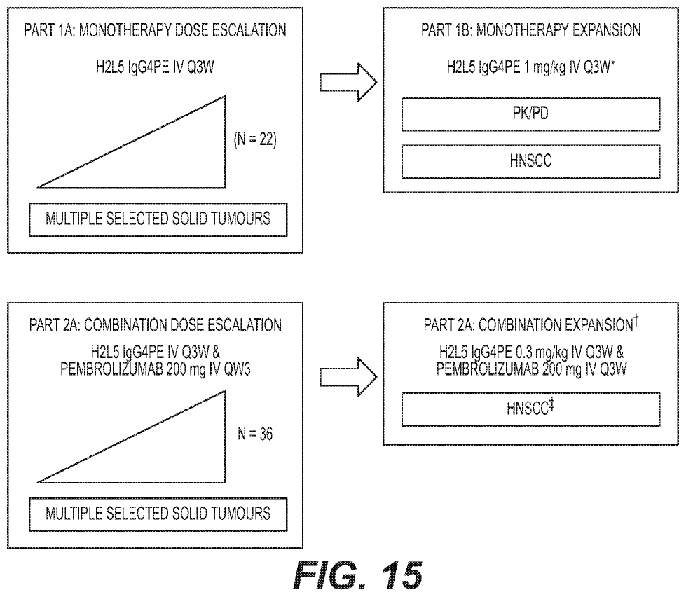

Within the HNSCC cohorts, the recommended doses from Parts 1A and 2A were selected for further investigation of safety, PK, PD activity, and preliminary clinical activity in Part 1B and 2B (combination expansion (CE)) (FIG. 15).
Disease assessments were performed every 9 weeks through Week 54 then every 12 weeks
Overall response rate (ORR), disease control rate (DCR) and progression-free survival (PFS) were assessed.

### Results

### Demographics

As of 26 Jul. 2019, 17 PD-1/L1-experienced patients in the monotherapy and 34 PD-1/L1-naïve patients in the combination HNSCC expansion cohort (EC) were enrolled; 16 and 34 patients respectively were evaluable for efficacy analysis (evaluable population comprises all participants who received 1 dose of H2L5 IgG4PE and had ≥1 post-baseline disease assessment, or had progressed, died or permanently discontinued treatment).
In the monotherapy EC, 82% of patients in the all-treated population (patients who received ≥1 dose of H2L5 IgG4PE) received ≥1 prior lines in the metastatic setting; in the combination EC, 53% received ≥1 prior lines in the metastatic setting.

TABLE 4
Patient demographics and disease characteristics

| | Monotherapy | Combination CE |
|---|---|---|
| | CE N = 17 | N = 34 |
| Sex, n (%), female/male | 3 (18)/14 (82) | 5 (15)/29 (85) |
| Median age, years (min, max) | 56 (41-73) | 62 (37-77) |
| Primary site, n (%) | | |
| Oropharynx | 5 (29) | 9 (26) |
| Non-oropharynx | 12 (71) | 25 (74) |
| Disease setting, n (%) | | |
| Metastatic | 16 (94) | 31 (91) |
| Local/regional recurrent only | 1 (6) | 3 (9) |
| ≥2 target & non-target lesion | 16 (94) | 27 (79) |
| locations, n (%) | | |
| ≥3 prior systemic regimens in | 6 (35) | 2 (6) |
| advanced/metastatic setting, n (%) | | |

### Efficacy

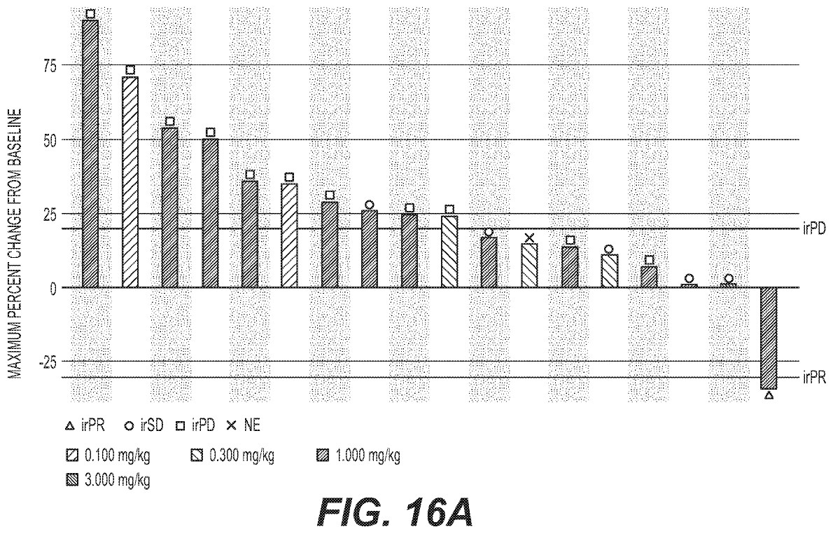

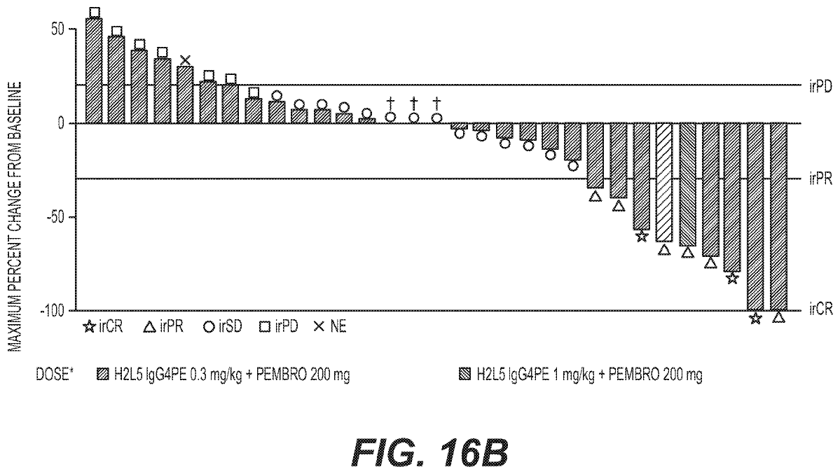

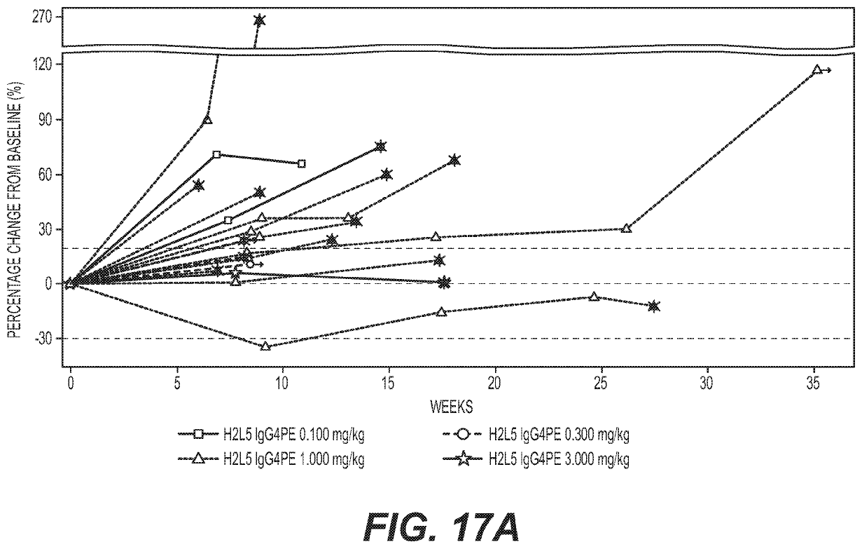

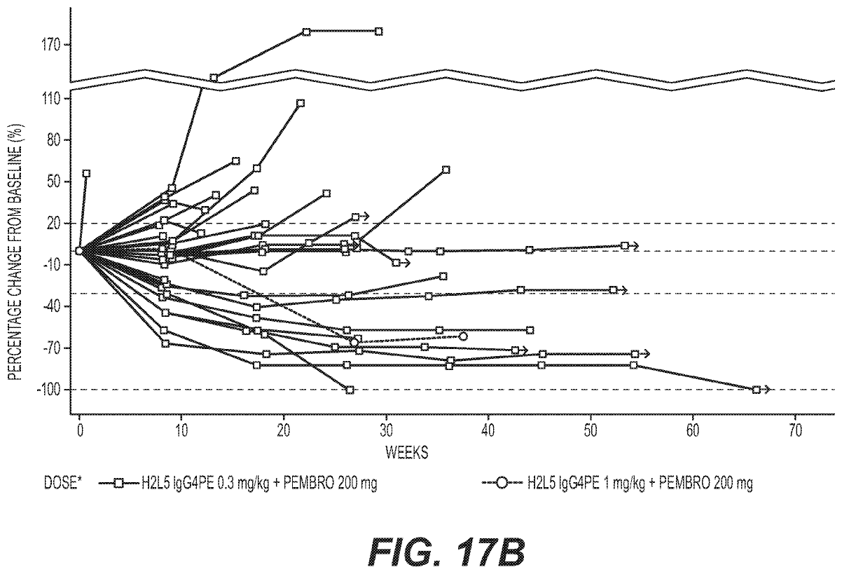

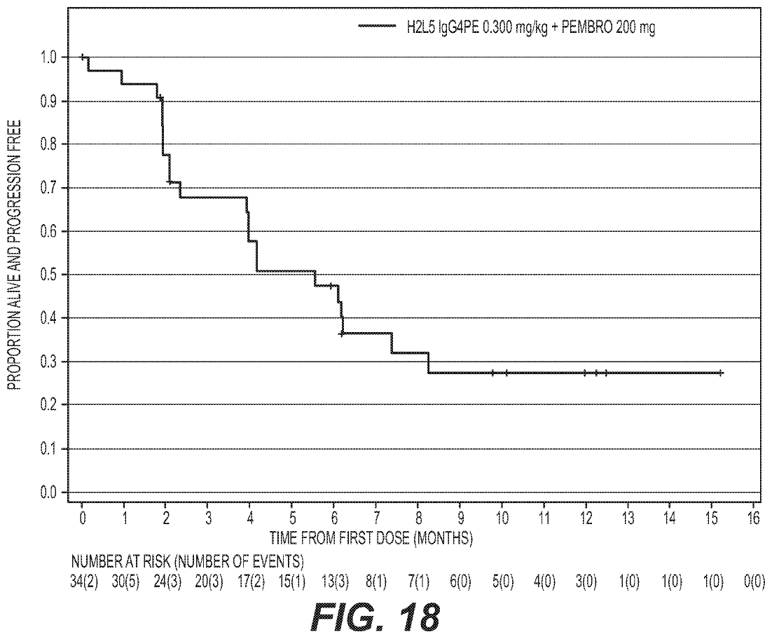

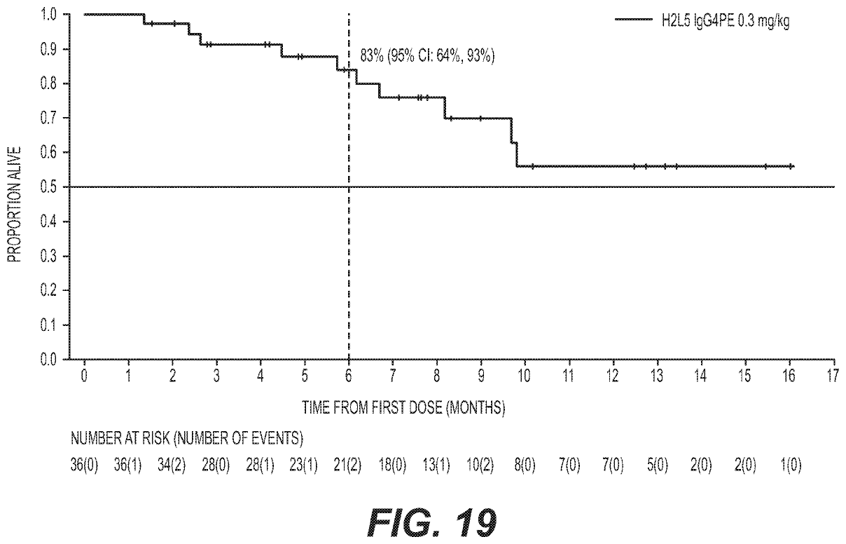

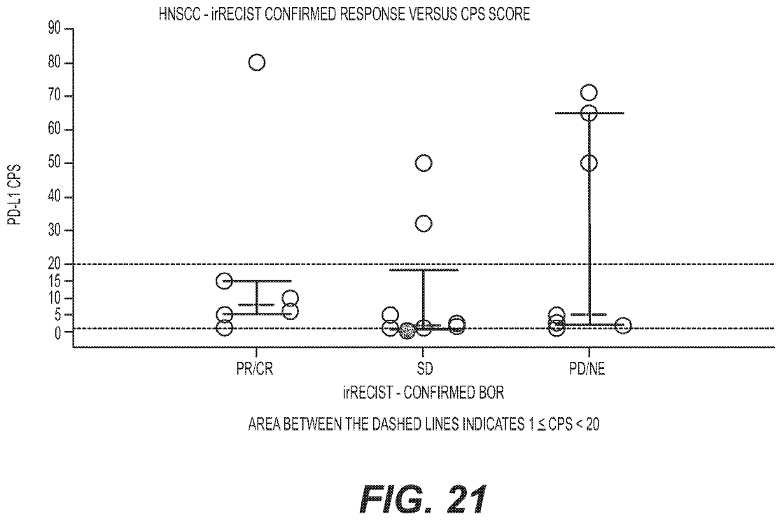

Of the 16 evaluable patients in the monotherapy cohort, the overall response rate (ORR) was 6% (95% CI: 0.2, 30.2) and the disease control rate (DCR)), defined as the percentage of patients with a complete response, partial response or stable disease for ≥9 weeks, was 31% (95% CI: 11, 58.7) (FIG. 16A). In the combination cohort ORR was 24% (95% CI: 10.7, 41.2) (FIG. 16B) and the DCR was 65% (95% CI: 46.5, 80.3) from 34 evaluable patients.
Responses in the combination cohort were durable with all responding patients maintaining benefit for ≥6 months (median not reached (NR); 95% CI [4.2 months, NR](FIG. 17B).
Median PFS in the combination cohort was 5.6 months (95% CI: 2.4, 7.4) (FIG. 18).
For the combination cohort, median OS was not reached at time of analysis (95% CI: 8.2, NR) (FIG. 19); the Kaplan-Meier estimate of OS at 6 months was 83% (95% CI: 64%, 93%).
PD-L1 immunohistochemistry testing with DAKO 22C3 is ongoing (FIG. 21). Among the patients with known PD-L1 data, a majority of responders and patients with stable disease have PD-L1 status 1≤CPS <20 (10/14 patients with CPS ≥1 and <20, and I patient with CPS<1).

### Safety

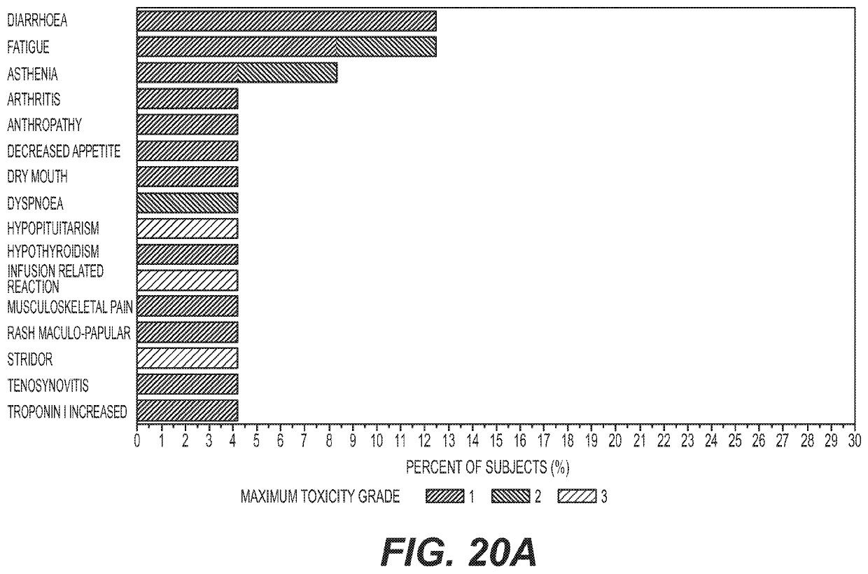

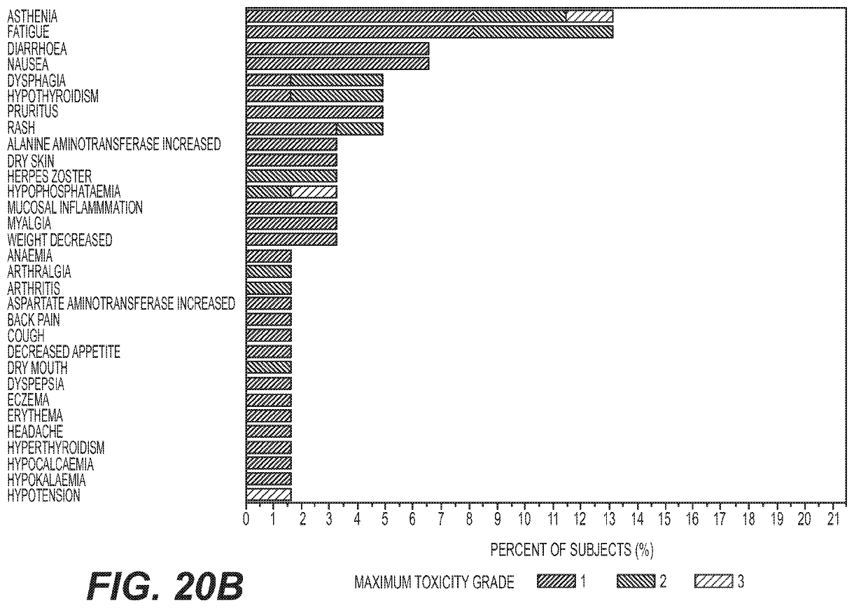

Treatment-related adverse events in patients with HNSCC across all study cohorts in the monotherapy and combination populations (FIG. 20) were consistent with that previously reported and both H2L5 IgG4PE alone and in combination with pembrolizumab was well tolerated.
Adverse events (AEs) and serious AEs (SAEs) in patients with HNSCC (all study cohorts) are described in Table 5.

TABLE 5
AEs and SAEs in the HNSCC cohort

| | Monotherapy | Combination |
|---|---|---|
| | H2L5 | H2L5 |
| | IgG4PE | IgG4PE all doses |
| | all | & pembrolizumab |
| | doses | 200 mg |
| n, (%) | (N = 22) | (N = 58) |
| Any AE | 22 (100) | 52 (90) |
| AE related to study treatment | 9 (41) | 37 (64) |
| AEs leading to permanent | 0 | 3 (5) |
| discontinuation of study treatment | | |
| AEs leading to dose interruption/delay | 3 (14) | 5 (9) |
| Any Grade 3 or 4 AE | 15 (68) | 17 (29) |
| Grade 3 or 4 related to study treatment | 2 (9) | 4 (7) |
| Grade 5 AEs | 0 | 2 (3) |
| Grade 5 related to study treatments | 0 | 0 |

### Patient Case Study

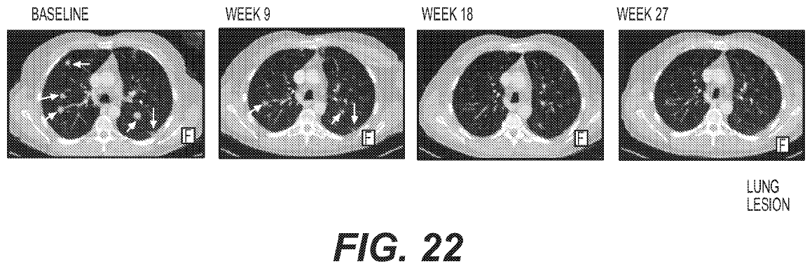

### H2L5 IgG4PE/Pembrolizumab Combination Therapy: HNSCC Patient—61Y Male (FIG. 22)

### History:

### Diagnosis (Dx):

Initial Dx: November 2017, Stage III, HPV+ oropharyngeal squamous cell carcinoma
Dx with metastasis: December 2017

### Prior Regimens:

Radiotherapy (January-February 2018, progressive disease [PD])
Cisplatin (radiosensitiser, January 2018)
Carboplatin/paclitaxel (radiosensitiser, 20 Feb. 2018)

### Study Treatment:

C1D1: 6 Aug. 2018; discontinued after Week 30 due to progressive disease (PD)
H2L5 IgG4PE at 0.3 mg/kg Q3W+200 mg pembrolizumab Q3W

### Post Treatment Sample Showed:

More tumour infiltrating lymphocytes including cytotoxic T cells, helper T cells and NK cells.
More proliferating T cells, granzyme B+ cells and less proliferating tumour cells.
More activated T cells as observed with greater OX40 and human leukocyte antigen DR expression.

FIG. 22 shows CT images from baseline (prior to initiation of H2L5 IgG4PE/pembrolizumab) and at on-study treatment assessment intervals of lung lesions showing lesion responses at Week 9 that were durable as evidenced by subsequent assessments.

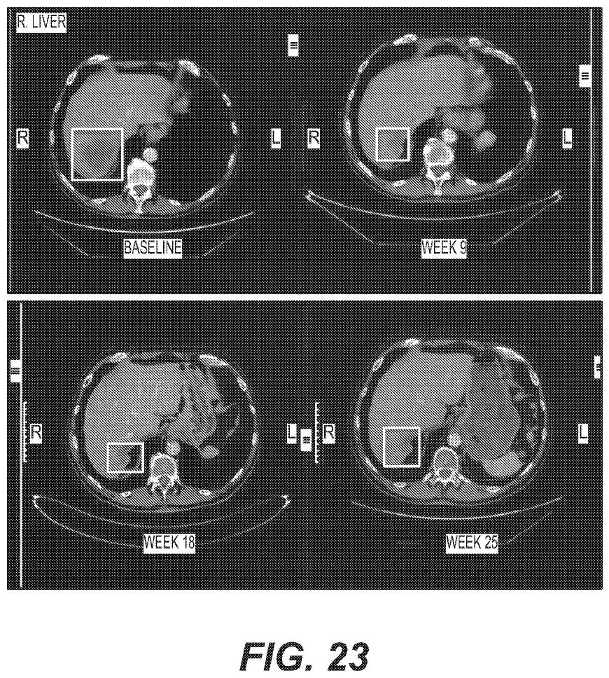

### H2L5 IgG4PE Monotherapy: HNSCC Patient—64Y Male (FIG. 23)

### History:

### Diagnosis (Dx):

Initial Dx: April 2013; Stage Iva HNSCC (oral cavity)

### Prior Regimens:

Cisplatin/radiotherapy (adjuvant, July-September 2013, complete response)
Carboplatin (December 2016-March 2017; progressive disease)→cetuximab maintenance (March-May 2017; progressive disease)
Methotrexate (June-August 2017; progressive disease)
Nivolumab (October-December 2017; progressive disease)

### Study Treatment

Cycle 1 Day 1 (C1D1): 15 Jan. 2018
H2L5 IgG4PE monotherapy at 1 mg/kg Q3W to Week 30, then receiving H2L5 IgG4PE+200 mg pembrolizumab Q3W (crossover) from Week 36 to Week 51
FIG. 23 shows CT images from baseline (prior to initiation of H2L5 IgG4PE monotherapy) and at on-study treatment assessment intervals of liver lesion showing a near 50% decrease in lesion size at Week 9 that was durable as evidenced by subsequent assessments.

### Conclusions

H2L5 IgG4PE monotherapy and in combination with pembrolizumab shows a manageable safety profile in patients with previously treated, PD-1/L1-naïve HNSCC.
H2L5 IgG4PE has demonstrated both single agent activity in PD-1/L1-experienced HNSCC and activity in combination with pembrolizumab in PD-1/L1 naïve HNSCC; median PD-L1 expression was lower in patients with progressive disease compared with those with stable disease and those with CR/PR.
Clinical translational work supports the continuous exploration of H2L5 IgG4PE as monotherapy and in combination with pembrolizumab in HNSCC.

1. A method of treating cancer in a human in need thereof, the method comprising administering to the human an agonist ICOS binding protein or antigen binding portion thereof at a dose of about 0.08 mg to about 240 mg.

2-3. (canceled)

4. A pharmaceutical kit comprising an ICOS binding protein or antigen binding portion thereof at a concentration of 10 mg/ml.

5. The method of claim 1, wherein the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5; CDRL3 as set forth in SEQ ID NO:6 and/or one or more of CDRH1, CDRH2, CDRH3, CDRL1, CDRL2, and CDRL3 that has no more than two amino acid substitutions.

6. The method of claim 1, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS.

7. The method of claim 1, wherein the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6.

8. The method of claim 1, wherein the ICOS binding protein comprises a V_{H} domain comprising the amino acid sequence set forth in SEQ ID NO:7 and a V_{L} domain comprising the amino acid sequence as set forth in SEQ ID NO:8.

9. The method of claim 1, wherein the ICOS binding protein comprises an hIgG4PE scaffold.

10. The method of claim 1, wherein the ICOS binding protein is a monoclonal antibody.

11. The method of claim 1, wherein the ICOS binding protein is a humanized monoclonal antibody.

12. The method of claim 1, wherein the ICOS binding protein is administered at a dose of 0.08 mg, 0.24 mg, 0.8 mg, 2.4 mg, 8 mg, 24 mg, 80 mg, or 240 mg.

13. The method of claim 1, wherein the ICOS binding protein is administered at a dose of 8 mg, 24 mg, or 80 mg.

14. The method of claim 1, wherein the ICOS binding protein is administered via IV infusion.

15. The method of claim 1, wherein the cancer is a solid tumor.

16. The method of claim 1, wherein the cancer is selected from the group consisting of: colorectal cancer, cervical cancer, bladder cancer, urothelial cancer, head and neck cancer, HNSCC, melanoma, mesothelioma, non-small cell lung carcinoma, prostate cancer, esophageal cancer, and esophageal squamous cell carcinoma.

17. The method of claim 1, wherein the ICOS binding protein is administered about once every three weeks.

18. A pharmaceutical formulation comprising an ICOS binding protein or an antigen binding portion thereof at a concentration of 10 mg/ml.
